(19) United States
(12) Patent Application Publication
(10) Pub. No.: US 2009/0004259 A1
(43) Pub. Date: Jan. 1, 2009

(54) METHODS OF PREPARING A THERAPEUTIC FORMULATION COMPRISING GALECTIN-INDUCED TOLEROGENIC DENDRITIC CELLS

(75) Inventors: Gabriel Adrian Rabinovich, Buenos Aires (AR); Juan Martin Ilarregui, Buenos Aires (AR); Marta Alicia Toscano, Buenos Aires (AR); German Ariel Bianco, Buenos Aires (AR)

Correspondence Address: YOUNG & THOMPSON, 209 Madison Street, Suite 500, ALEXANDRIA, VA, 22314, US

(73) Assignees: CONSEJO NACIONAL DE INVESTIGACIONES CIENTIFICAS Y TECNICAS (CONICET), BUENOS AIRES (AR); FUNDACION SALES, BUENOS AIRES (AR)

(21) Appl. No.: 12/137,004

(22) Filed: Jun. 11, 2008

Related U.S. Application Data
(60) Provisional application No. 60/934,842, filed on Jun. 14, 2007.

(51) Int. Cl. A61K 9/127; A61K 35/12; A61K 9/14

(52) U.S. Cl. 424/450; 424/93.7; 424/489

(57) ABSTRACT

In spite of their pivotal role in orchestrating immunity, dendritic cells (DCs) may be licensed by immunosuppressive signals to become tolerogenic. Here we show that ligation of cell surface glyco-receptors by Galectin-1, an endogenous glycan-binding protein, can drive the differentiation of regulatory DCs with tolerogenic potential in vivo. Galectin-1-differentiated DCs acquired a tolerogenic phenotype characterized by IL-27-dependent, STAT3-mediated and CD45RB⁺IL-10⁺ regulatory signatures. Adoptive transfer of galectin-1-conditioned DCs induced T-cell tolerance in inflammatory and neoplastic settings and dampened T_{H}1- and T_{H}-17-mediated autoimmune neuroinflammation. Consistent with a negative regulatory function of endogenous galectin-1, DCs from galectin-1-deficient (Lgals1^{-/-}) mice had greater immunogenic capacity compared with their wild-type counterparts. Our findings identify a crucial role of galectin-1 in the differentiation of IL-27-producing tolerogenic DCs with broad therapeutic implications in immunopathology. Thus, the present invention encompasses therapeutic formulations, comprising Galectin-induced tolerogenic DCs and a therapeutical acceptable carrier, methods of preparing said formulations and methods of using same.

This application claims priority to U. S. provisional application no. US60/934,842, filed Jun. 14, 2007, the entirety of which is incorporated herein by reference.

### 1. FIELD OF THE INVENTION

This invention relates to a method of preparing a therapeutic formulation which comprises incubating dendritic cells (DCs) or dendritic cells progenitors (DCPs) in a incubation medium containing galectin, wherein said galectin is in a sufficient amount for obtaining Galectin-induced tolerogenic DCs, and suspending said Galectin-induced tolerogenic DCs in a therapeutical acceptable carrier. This invention further encompasses a therapeutic formulation that comprises Galectin-induced tolerogenic DCs and a therapeutical acceptable carrier, and methods of using said formulation.

### 2. BACKGROUND

Dendritic cells (DCs) are highly specialized antigen-presenting cells (APCs) that recognize, process and present antigens to naïve T cells¹⁻³. For a long time, attention has been focused on the ability of these professional APCs to elicit T cell responses¹; however evidence has emerged concerning the ability of DCs to induce peripheral tolerance by promoting T cell anergy or favoring the differentiation of regulatory T cells, including CD4⁺CD25⁺Foxp3⁺ regulatory T cells (Tregs) and T regulatory type-1 (Tr1) cells³⁻⁵.

Several factors may influence the decision of DCs to become immunogenic or tolerogenic, including their cytokine milieu at sites of inflammation, infection or tumor growth³⁻⁵. Human or murine DCs can be endowed with tolerogenic potential by cytokines, neuropeptides and growth factors including interleukin (IL)-10 (Ref. 6), vasoactive intestinal peptide⁷, hepatocyte growth factor⁸ and 1,25-dihydroxyvitamin D3 (Ref. 9). Furthermore, engagement of cell surface receptors by apoptotic cells¹⁰, or interaction with stromal cells^{11,12} may also program the differentiation of human or mouse regulatory DCs, which in turn foster the expansion of Tr1 cells. Of interest, recent evidence indicates that DCs modified by CD4⁺CD25⁺ Tregs may become tolerogenic and drive the differentiation of IL-10-producing Tr1 cells through an IL-27-dependent mechanism¹³⁻¹⁵, suggesting an important link between distinct regulatory cell populations.

During the past few years, there has been increasing appreciation for the impact of protein-glycan interactions in the regulation of innate and adaptive immune responses¹⁶. Galectin-1, a glycan-binding protein up-regulated at sites of inflammation and tumor growth, elicits a broad spectrum of biological functions predominantly acting as a potent anti-inflammatory factor and a suppressive agent for T-cell responses¹⁷⁻²¹. Blockade of galectin-1 expression in tumor tissue results in heightened T cell-mediated tumor rejection and increased secretion of T_{H}1-type cytokines^{22,23}. In addition, galectin-1-deficient (Lgals1^{-/-}) mice show greater T_{H}1 and T_{H}-17 responses and are considerably more susceptible to immune-mediated fetal rejection and autoimmune disease than their wild-type counterparts^{21,24}, suggesting an essential role for this glycan-binding protein in the control of immune tolerance and homeostasis.

Because DCs are pleiotropic modulators of T-cell activity and are endowed with exquisite plasticity, manipulation of their function, to favor the induction of DCs with tolerogenic properties could be exploited to attenuate immune responses, particularly for the control of autoimmune diseases and graft rejection^{4,5}. In sharp contrast, overcoming immune tolerance by depletion of tolerogenic DCs or by silencing negative regulatory signals might have selective advantages for the success of tumor immunotherapy strategies²⁵.

### 3. SUMMARY OF THE INVENTION

The inventors of the present patent here show, by a combination of in vitro and in vivo strategies, that galectin-1-glycan lattices can drive the differentiation of human and mouse tolerogenic DCs, which negatively regulate T cell responses through IL-27-dependent and STAT3-mediated mechanisms. These tolerogenic DCs promoted antigen-specific tolerance in inflammatory and neoplastic settings and dampened T_{H}1 and T_{H}17-mediated autoimmune inflammation. In addition, Lgals1^{-/-} DCs had greatly enhanced immunogenic capacity compared with their wild-type counterparts, suggesting a critical role of endogenous galectin-1 in ‘fine-tuning’, the tolerogenic function of these cells. Thus, modulation of galectin-1 expression or its specific carbohydrate ligands within the DC compartment may provide novel opportunities for therapeutic harnessing of the inherent tolerogenicity of DCs.

The present invention encompasses a method of preparing a therapeutic formulation which comprises incubating dendritic cells (DCs) or dendritic cells progenitors (DCPs) in a incubation medium containing galectin, wherein said Galectin is in a sufficient amount for obtaining Galectin-induced tolerogenic DCs; and suspending said Galectin-induced tolerogenic DCs in a therapeutical acceptable carrier. In a particular embodiment of the invention, said therapeutical acceptable carrier is a pharmaceutical acceptable excipient, vehicle and/or diluent. Preferably, the method of the invention comprises incubating dendritic cells (DCs) in a incubation medium containing Galectin-1 or Galectin-2, wherein said Galectin is in a sufficient amount for obtaining Galectin-induced tolerogenic DCs. In a more preferred embodiment, said incubation medium contains Galectin-1. Preferably, said incubation medium containing Galectin, contains from about 0.1 to about 10 μM of Galectin-1 and more preferably contains from about 0.3 to 3/M of Galectin-1.

Following the method of preparing a therapeutic formulation of the invention, Galectin-induced tolerogenic DCs acquire a tolerogenic phenotype characterized by IL-27-dependent, STAT3-mediated and CD45RB⁺IL-10⁺ regulatory signatures.

It is another object of the invention a therapeutic formulation, comprising Galectin-induced tolerogenic DCs and a therapeutical acceptable carrier and methods of treating, managing or preventing several diseases or disorders.

### 4. BRIEF DESCRIPTION OF THE FIGURES

FIG. 1. Galectin-1 interferes with the differentiation and function of human DCs. (a-d) Analysis of human monocyte-derived DCs differentiated for 7 d with recombinant human GM-CSF and IL-4 in the absence (iDC) or presence (iDC_{Gal-1}) of galectin-1 (3 μM unless stated otherwise). (a) Flow cytometry of phenotypic makers of iDC or iDC_{Gal-1} (left; thick lines, nonspecific binding determined with isotype-matched control antibodies; gray shading, differentiation markers). Data are from one representative of eight independent experiments. Numbers in parentheses represent the relative mean fluorescence intensity (rMFI): (MFI specific antibody−MFI isotype control)/MFI isotype control. Right, dose- and carbohydrate-dependent modulation of CD14 and CD1a expression. *, P<0.05. (b) Flow cytometry analysis of endocytosis of FITC-OVA by iDC and iDC_{Gal-1} (left; thick lines, control at 4° C.; gray shading, 37° C.). Right, time-course study. (rMFI): MFI at 37° C.−MFI at 4° C.)/MFI at 4° C. *, P<0.05. Data are the mean±s.e.m. of four experiments. (c) [³H]-thymidine incorporation by allogeneic CD4 T cells cultured for 5 d with iDC or iDC_{Gal-1} (DC:T cell ratio, 1:5). **, P<0.01. Data are the mean+s.d. of five experiments. (d) ELISA of IFN-γ in supernatants of allogeneic CD4 T cells cultured for 5 d with iDC or iDC_{Gal-1}. **, P<0.01. Data are the mean+s.d. of five experiments. (e-i) Analysis of mature DCs cultured for 24 h with LPS in the absence (DC) or presence (DC_{Gal-1}) of galectin-1. (e) Flow cytometry of phenotypic markers of mature DC or DC_{Gal-1} (thick lines, nonspecific binding determined with control isotype antibodies; gray shading, maturation markers). Data are from one representative of six independent experiments. (f) Laser confocal microscopy of immature DCs incubated with galectin-1 or buffer control, fixed and stained with FITC-conjugated anti-human CD43 antibody and propidium iodide. Scale bar, 20 μm (insets, 5 μm). Percent CD43 segregation is shown in Supplementary FIG. 1c online. (g) ELISA of IL-12p70 (left) and IL-10 (right) in supernatants of DCs matured in the absence or presence of galectin-1. *, P<0.05. Data are the mean+s.d. of four experiments. (h) [³H]-thymidine incorporation by allogeneic CD4 T cells cultured for 5 d with mature DC or DC_{Gal-1}. **, P<0.01. Data are the mean+s.d. of four experiments. (i) ELISA of IFN-γ (left) and IL-10 (right) in supernatants of allogeneic CD4 T cells cultured for 5 d with mature DC or DC_{Gal-1}.*, P<0.05; **, P<0.01. Data are the mean+s.d. of four experiments.

FIG. 2. Galectin-1 imparts a regulatory program in human mature DCs. (a-c) Analysis of human allogeneic CD4 T cells co-cultured for 5 d with LPS-matured DCs (DC; 1×10⁴) in the absence or presence of variable numbers of galectin-1-matured DCs (DC_{Gal-1}). (a) [³H]-thymidine incorporation by allogeneic CD4 T cells. *, P<0.05; **, P<0.01. Data are the mean+s.d. of four experiments. (b,c) ELISA of IFN-γ (b) and IL-10 (c) in supernatants of allogeneic CD4 T cells. *, P<0.05; **, P<0.01. Data are the mean+s.d. of four experiments. (d) Immunoblot analysis of pSTAT3 on DCs matured in the absence (DC) or presence (DC_{Gal-1}) of galectin-1; relative expression (RE), band intensity relative to that of STAT3. Data are representative of three experiments. (e) Analysis of the allostimulatory capacity of human DCs matured with Gal-1 in the absence (DC_{Gal-1}) or presence (AG490-DC_{Gal-1}) of variable doses of the JAK2-STAT3 inhibitor AG490. [³H]-thymidine incorporation by human allogeneic CD4 T cells co-cultured for 5 d with fully competent DCs (DC), DC_{Gal-1} or AG490-DC_{Gal-1}. **, P<0.01; ***, P<0.001. Data are the mean+s.d. of three independent experiments.

FIG. 3. Galectin-1 programs the differentiation of CD45RB⁺IL-27^{hi} mouse tolerogenic DCs. Analysis of bone marrow-derived DCs differentiated for 9 d with recombinant mouse GM-CSF in the absence (DC) or presence (DC_{Gal-1}) of galectin-1 (3 μM). (a) Flow cytometry of phenotypic markers of DC or DC_{Gal-1} (left; thick lines, nonspecific binding determined with isotype-matched control antibodies; gray shading, differentiation markers). Data are from one representative of nine independent experiments. Numbers in parentheses represent the rMFI: (MFI specific antibody−MFI isotype control)/MFI isotype control. Right, carbohydrate-dependent modulation of CD11c and CD45RB expression. *, P<0.05; **, P<0.01; ***, P<0.001. (b) Real time quantitative RT-PCR analysis of the expression of IL-27p28 on DC and DC_{Gal-1}; fold increase relative to the expression of mRNA encoding GAPDH. *, P<0.05. Data are the mean+s.d. of three experiments. (c-e) ELISA of IL-6 (c), IL-10 (d), IL-12p70 (e) in supernatants of DCs differentiated in the absence (DC) or presence (DC_{Gal-1}) of galectin-1 and further matured with LPS. (f) [³H]-thymidine incorporation by BALB/c CD4 splenocytes stimulated for 5 d with B6 DC or DC_{Gal-1} (DC:T ratio, 1:10) in the absence or presence of neutralizing antibodies (Ab) specific for IL-27p28, TGF-β or IL-10 receptor (IL-10R). **, P<0.01. Data are the mean+s.d. of four experiments. (g) ELISA of IFN-γ (left), IL-17 (middle) and IL-10 (right) in supernatants of BALB/c CD4 splenocytes stimulated for 5 d with DC or DC_{Gal-1} in the presence or absence of a neutralizing antibody specific for IL-27p28. *, P<0.05; **, P<0.01. Data are the mean+s.d. of four experiments. (h) Analysis of the allostimulatory capacity of mouse DCs matured with galectin-1 in the absence (DC_{Gal-1}) or presence (AG490-DC_{Gal-1}) of the STAT3 inhibitor AG490. [³H]-thymidine incorporation by BALB/c CD4 splenocytes co-cultured for 5 d with B6 fully competent DCs, DC_{Gal-1} or AG490-DC_{Gal-1}. *, P<0.05. Data are the mean+s.d. of three independent experiments. (i) Immunoblot analysis of pSTAT3 on DCs matured in the absence (DC) or presence (DC_{Gal-1}) of galectin-1; bottom: relative expression (RE), band intensity relative to that of actin. Data are representative of three experiments.

FIG. 4. Galectin-1-differentiated IL-27-producing DCs induce antigen-specific tolerance in vivo. (a-d) Bone marrow-derived DCs differentiated in the absence or presence of galectin-1 were pulsed with OVA (OVA-DC or OVA-DC_{Gal-1}) and transferred into syngeneic mice. Seven days after transfer, mice were immunized with OVA in CFA and one week later splenocytes were collected, restimulated ex vivo with OVA or KLH and analyzed for antigen-specific proliferation and cytokine secretion. (a) [³H]-thymidine incorporation. ***, P<0.001. Data are the mean+s.d. of three independent experiments with four to five mice per group. (b-d) ELISA of IFN-γ (b), IL-17 (c) and IL-10 (d) in supernatants of antigen-restimulated splenocytes. *, P<0.05. Data are the mean+s.d. of three experiments with four to five mice per group.

FIG. 5. Galectin-1-differentiated DCs have impaired antitumor activity and foster a tolerant microenvironment at tumor sites. (a) Kinetics of tumor growth of B6 mice immunized with tumor lysate-pulsed DCs (Lys-DC), tumor-lysate pulsed DC_{Gal-1} (Lys-DC_{Gal-1}) or vehicle control twice at 7-d intervals and further challenged with viable B16 melanoma cells. Data represent the mean±s.e.m. of three experiments with five to six mice per group. *, P<0.05; **, P<0.01. Tumor growth of mice immunized with unpulsed DC is shown as Supplementary FIG. 5 online. (b) Kaplan Meier analysis of mice immunized with Lys-DC, Lys-DC_{Gal-1} or vehicle control and further challenged with viable B16 melanoma cells. *, P<0.05 Lys-DC versus Lys-DC_{Gal-1}. (c-e) Proliferative response and cytokine production in tumor-draining lymph node cells analyzed two weeks after tumor challenge following restimulation for 72 h with irradiated B16 cells. (c) [H³]-thymidine incorporation. **, P<0.01. Mean values of different groups are indicated (mean+s.d.) as combination of three independent experiments. (d,e) ELISA of IFN-γ (d) and IL-10 (e) in supernatants of lymph node cells from different groups of mice. **, P<0.01. Mean values of different groups are indicated (mean+s.d.) as combination of three independent experiments.

FIG. 6. Therapeutic administration of galectin-1-conditioned DCs halts autoimmune neuroinflammation and dampens T_{H}-17 and T_{H}1 responses. (a) Disease progression in mice immunized with MOG(35-55) and treated with MOG(35-55)-pulsed DC (MOG-DC) or MOG(35-55)-pulsed DC_{Gal-1} (MOG-DC_{Gal-1}). Arrow indicates the time of DC injection. **, P<0.01. Data are the mean±s.e.m. of three experiments with six mice per group. Disease progression in mice immunized with unpulsed DCs is shown as Supplementary FIG. 6a online. (b) Histopathology of spinal cord sections from mice treated with MOG-DC or MOG-DC_{Gal-1}, stained with hematoxilin and eosin (H&E) or Luxol Fast blue. Arrows indicate inflammatory infiltrates and demyelinated areas. Scale bars, 25 μm. Right columns are amplifications of the dotted square from left column. Data are representative of three experiments. (c) MOG(35-55)-specific proliferation analyzed by [³H]-thymidine incorporation of splenocytes from different groups of mice at 25 d after immunization. **, P <0.01. Data are the mean+s.d. of three experiments with six mice per group. (d-f) ELISA of IL-17 (d), IFN-γ (e) and IL-10 (f) in supernatants of splenocytes obtained at 25 d after immunization and restimulated ex vivo with MOG(35-55) for 72 h. *, P<0.05; **, P<0.01. Data are the mean+s.d. of three experiments with six mice per group.

FIG. 7. Endogenous galectin-1 controls cytokine production and allostimulatory capacity of DCs. (a) Laser confocal microscopy of galectin-1 (Gal-1; green) and CD11c (red) expression in bone marrow-derived immature and mature DCs. Scale bars, 10 μm (insets: 5 μm). (b) Immunoblot analysis of galectin-1 in lysates from immature and mature DCs; relative expression (RE), band intensity relative to that of actin. Data are representative of three independent experiments. (c) Flow cytometry of MHC II (I-Ab) expression by bone marrow-derived Lgals1^{-/-} (DC^{-/-}) and wild-type (DC^{+/+}) DCs differentiated for 9 d with GM-CSF. Left; thick lines, nonspecific binding determined with isotype-matched control antibody; gray shading, I-A^{b}. Numbers in parentheses represent the relative mean fluorescence intensity (rMFI). Data are from one representative of four independent experiments grouped at the right. *, P<0.05. (d) Real time quantitative RT-PCR analysis of the expression of IL-27p28 on DC^{-/-} and DC^{+/+}; fold increase relative to expression of mRNA encoding GAPDH. *, P<0.05. Data are the mean+s.d. of three experiments. (e,f) ELISA of IL-12p70 (e) and IL-10 (f) in supernatants of DC^{-/-} and DC^{+/+} differentiated with GM-CSF and further matured with LPS. Data are the mean+s.d. of three experiments. (g,h) Allostimulatory capacity of DC^{-/-} and DC^{+/+} differentiated with GM-CSF and further matured with LPS. (g) [³H]-thymidine incorporation by allogeneic CD4 splenocytes (BALB/c) stimulated for 5 d with DC^{-/-} or DC^{+/+} (B6) (DC:T ratio, 1:10). *, P<0.05. Data are the mean+s.d. of four experiments. (h) ELISA of IFN-γ (left), IL-17 (middle) and IL-10 (right) in supernatants of CD4 splenocytes (BALB/c) stimulated for 5 d with DC^{-/-} or DC^{+/+} (B6). *, P<0.05. Data are the mean+s.d. of four experiments.

FIG. 8. Endogenous galectin-1 fine-tunes the tolerogenic function of DCs in vivo. (a-d). Bone marrow-derived Lgas11^{-/-} (DC^{-/-}) or wild-type (DC^{+/+}) DCs differentiated with GM-CSF were pulsed with OVA and transferred into either Lgas11^{-/-} (−/−) or wild-type (+/+) recipient mice. Seven days after transfer, mice were immunized with OVA in CFA and one week later splenocytes were restimulated ex viva with OVA and analyzed for proliferation (a) and cytokine secretion (b-d). (a) [³H]-thymidine incorporation by splenocytes from different groups of mice. *, P<0.05; **, P<0.01. Data are the mean+s.d. of three independent experiments with four to five mice per group. (b-d) ELISA of IFN-γ (b), IL-17 (c) and IL-10 (d) in supernatants of splenocytes from different groups of mice. *, P<0.05; **, P<0.01. Data are the mean+s.d. of three independent experiments with four to five mice per group.

### 5. DETAILED DESCRIPTION

### Definitions

Unless otherwise indicated, the term “dendritic cells” refers to cells of the mammalian immune system which main function is to capture and process antigen material and present it to other cells of the immune system called T cells, thus stimulating an adaptive immune responses; although they may also become tolerogenic under certain conditions and prevent the development of adaptive immune responses thus contributing to immune cell homeostasis.

Unless otherwise indicated, the term “galectin” refers to a glycan-binding protein that binds β-galactoside sugars attached to glycoproteins and glycocolipids on the surface of certain mammalian cells.

Unless otherwise indicated, “Galectin-induced tolerogenic DCs acquire a tolerogenic phenotype characterized by IL-27-dependent, STAT3-mediated and CD45RB⁺IL-10⁺ regulatory signatures” means that galectin-1-induced tolerogenic DCs have considerably higher expression of the cell surface marker CD45RB (34w versus 0.6% expression in non-tolerogenic control DCs) and express substantially higher amounts of IL-10 (0.6 ng/ml versus 0.3 ng/ml in non-tolerogenic control DCs) and increased levels of IL-27 mRNA (2 fold-increase compared to non-tolerogenic control DCs). These three parameters represent a reliable signature of a tolerogenic DC

### Results

Galectin-1 Interferes with the Differentiation and Function of Human DCs

In search for a potential mechanism responsible of the broad anti-inflammatory activity of galectin-1 in autoimmune diseases and in response to tumors¹⁷⁻²² we conducted an integrated study of the impact of galectin-1 on the differentiation and function of human and mouse DCs using different experimental strategies.

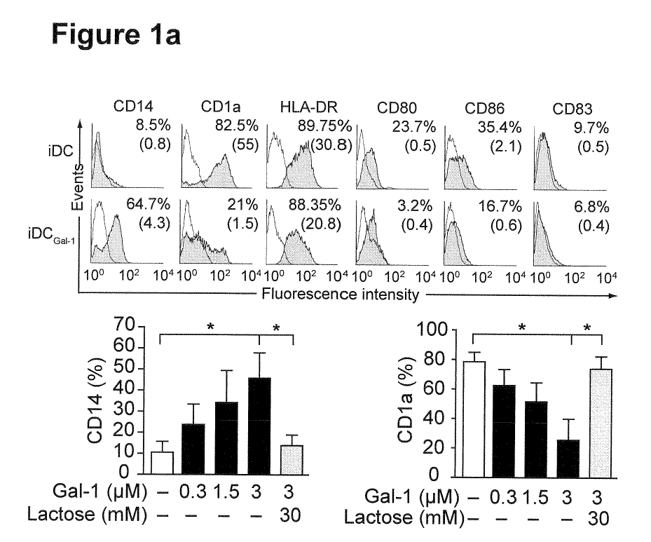

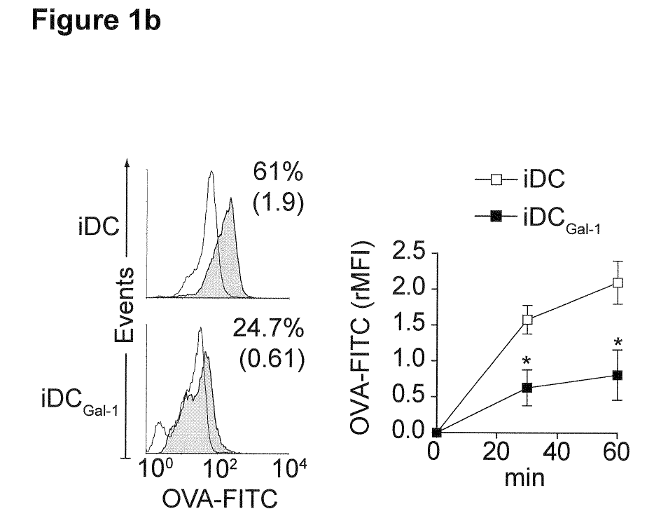

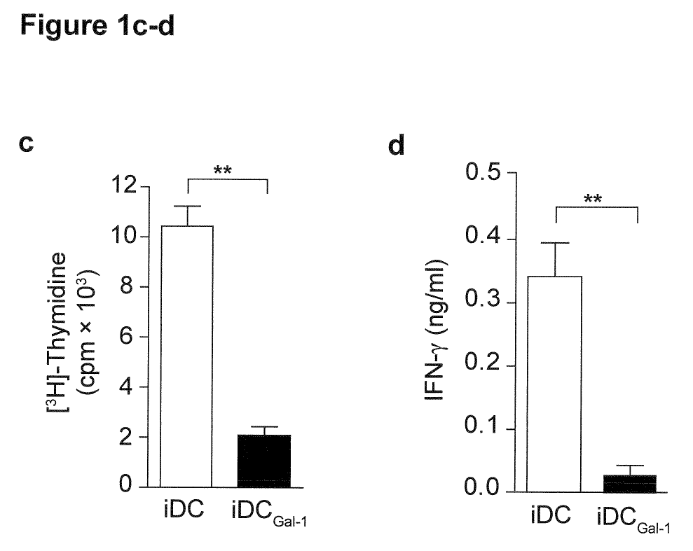

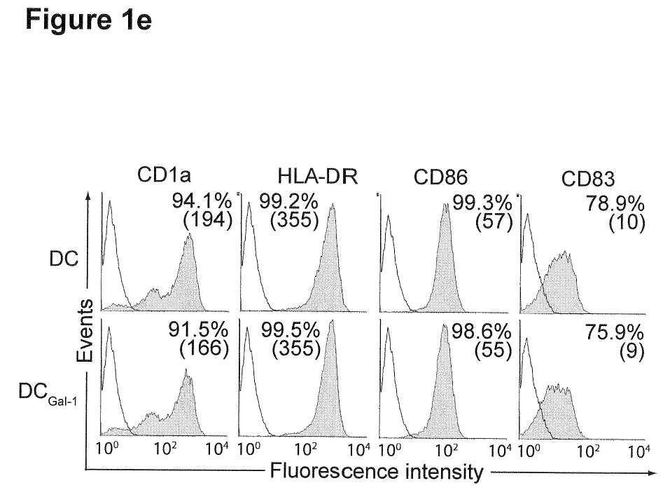

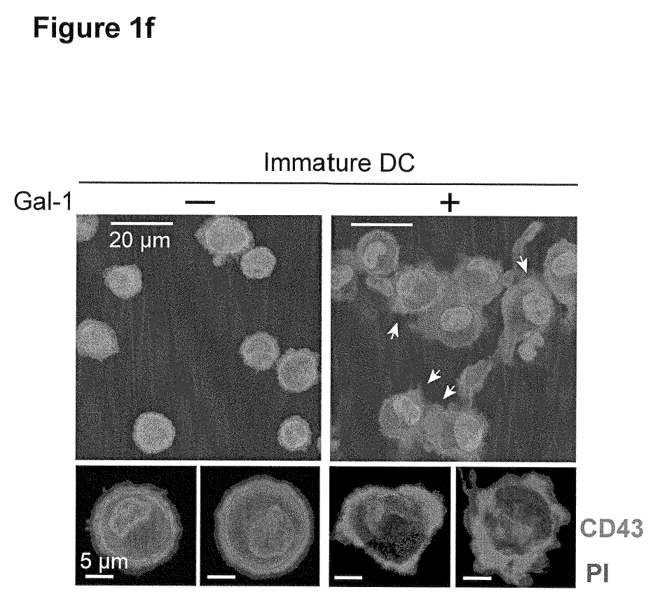

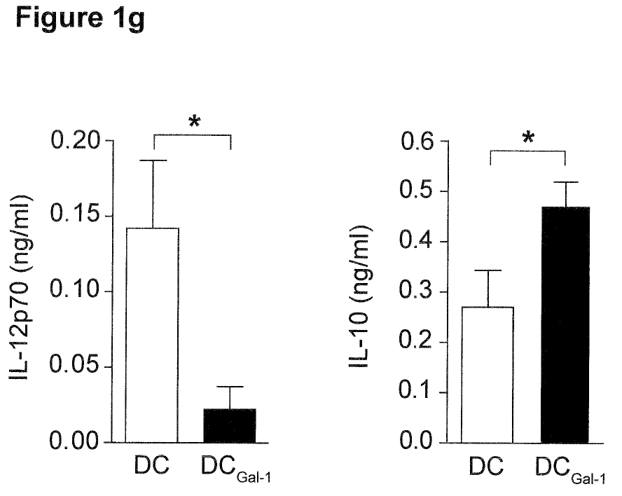

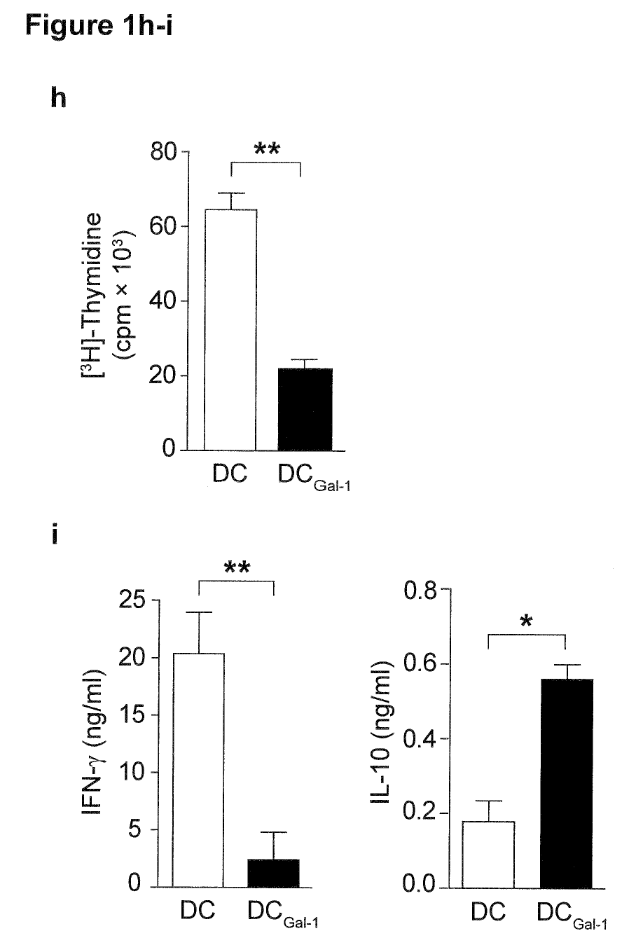

To determine whether exposure to galectin-1 during human DC differentiation results in phenotypic and functional changes, we first compared human monocyte-derived DCs generated in the absence or presence of galectin-1 (iDC_{Gal-1}) in terms of cell surface markers, endocytosis, cytokine production and allostimulatory capacity. Human monocytes differentiated with granulocyte-macrophage colony-stimulating factor (GM-CSF) and IL-4, showed the typical phenotypic markers of immature DCs (FIG. 1a). Galectin-1 bound to human monocytes in a dose- and carbohydrate-dependent manner (Supplementary FIG. 1a online), and largely interfered with the normal differentiation of immature DCs when added at the initiation of the cell culture, as reflected by the low expression of CD1a and the costimulatory molecules CD80 and CD86 and the substantial expression of CD14 in iDC_{Gal-1} compared to control iDC (FIG. 1a); this effect was prevented by co-incubation of galectin-1 with its specific disaccharide lactose (FIG. 1a, right panels). Furthermore, iDC_{Gal-1} exhibited lower capacity to endocytose FITC-labeled ovalbumin (OVA) compared to DCs differentiated in the absence of this protein (FIG. 1b). To examine the ability of galectin-1-differentiated DCs to prime and differentiate CD4 T cells, we co-cultured DC_{Gal-1} or control DCs with alloreactive CD4 T cells. Priming with control DCs resulted in substantial proliferation and considerable synthesis of interferon (IFN)-γ by allogeneic CD4 T cells, whereas DC_{Gal-1} induced only weak proliferation and negligible production of IFN-γ (FIGS. 1c,d). Thus, galectin-1 impairs the differentiation and allostimulatory capacity of human monocyte-derived DCs in vitro.

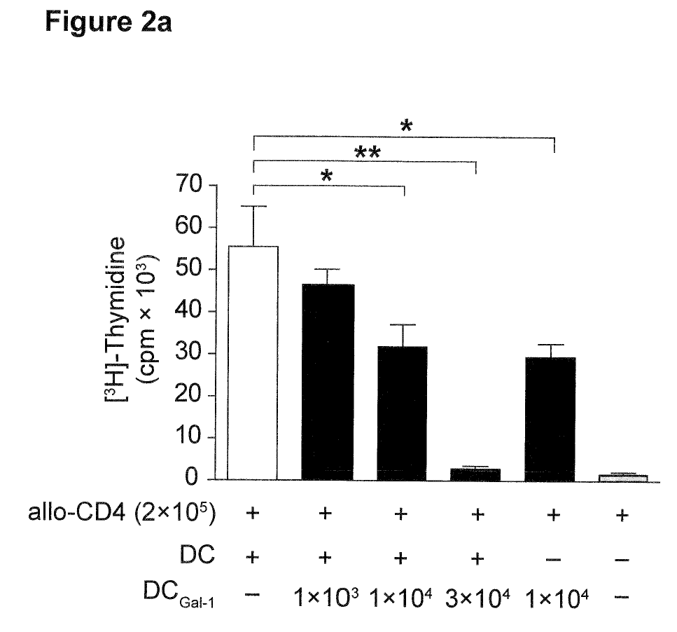

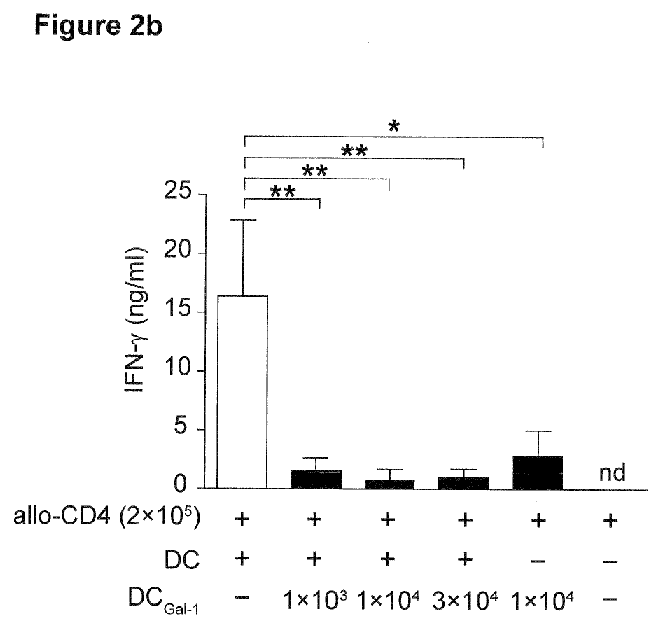

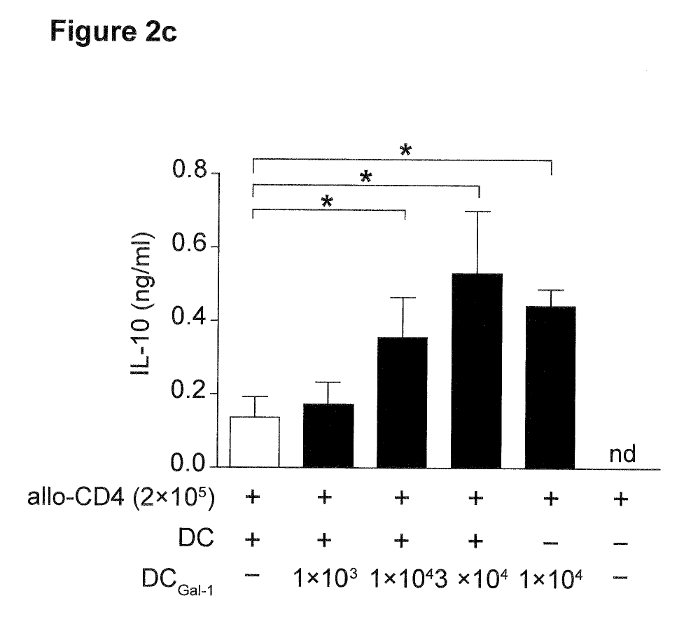

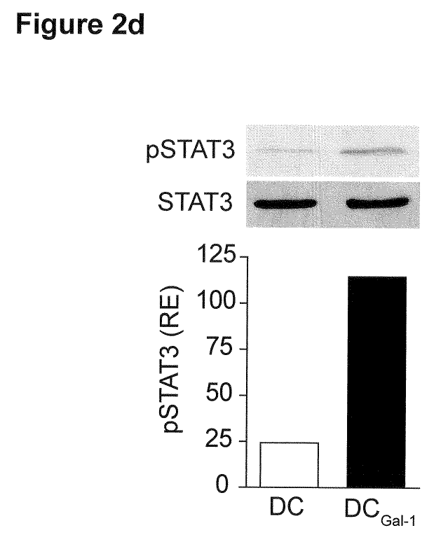

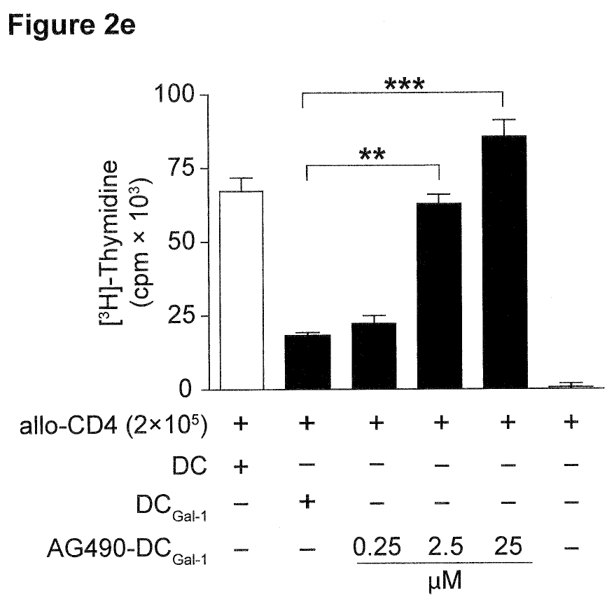

Upon Toll-like receptor engagement with lipopolysaccharides (LPS), immature human DCs mature into cells capable of expressing high levels of CD83, MHC II, and costimulatory molecules (FIG. 1e). We thus examined whether exposure to galectin-1 in the transition from immature to mature DCs may result in phenotypical or functional changes. As predicted by their permissive glycophenotype²⁶, immature DCs bound galectin-1 in a dose-dependent and carbohydrate-specific manner (Supplementary FIG. 1b online), and induced segregation of the glyco-receptor CD43, but not CD45 to membrane patches on human immature DCs (FIG. 1f and Supplementary FIG. 1c online). Remarkably, exposure to galectin-1 during LPS-induced DC maturation did not impinge on the resulting cell surface phenotype of mature DCs, which showed a similar profile to DCs matured with LPS alone (FIG. 1e). However, DCs matured in the presence of LPS and galectin-1 were capable of producing substantially higher amounts of IL-10 and lower amounts of IL-12, compared with those matured with LPS alone (FIG. 1g). Furthermore, in mixed leukocyte reactions (MLR), allogeneic CD4 T cells primed with galectin-1-conditioned DCs showed weak proliferation (FIG. 1h) and synthesized less IFN-γ and more IL-10 compared with allogeneic CD4 cells primed with control DCs (FIG. 1i). However, we could find no differences in the amounts of other cytokines including TGF-β or IL-4 in supernatants of MLR cultures (data not shown). Thus, regardless of a similar cell surface mature phenotype, exposure to galectin-1 overrides the capacity of LPS to induce IL-12-producing fully competent DCs. Of note, allogeneic CD4 T cells primed with DC_{Gal-1} did not show variations in the frequency of CD4⁺CD25⁺FoxP3⁺ cells, in spite of their capacity to suppress proliferation of activated T cells (Supplementary FIGS. 2a,b online).

To determine whether DCs matured in a galectin-1-enriched microenvironment had enhanced regulatory potential, we tested the capacity of these cells to inhibit the MLR stimulated by fully competent LPS-matured DCs. Galectin-1-conditioned DCs could suppress in a dose-dependent manner the MLR induced by fully competent DCs (FIG. 2a). This inhibitory effect was mirrored by a dramatic decline in IFN-γ (FIG. 2b) and a dose-dependent increase in IL-10 synthesis by alloreactive CD4 T cells (FIG. 2c). Thus, galectin-1 imparts a regulatory program on human DCs, which can effectively abrogate the T-cell allostimulatory capacity of fully competent DCs.

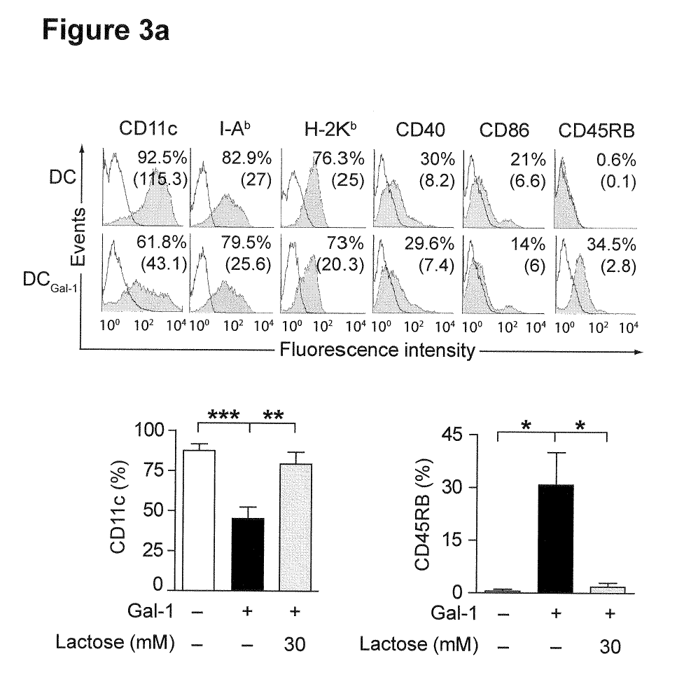

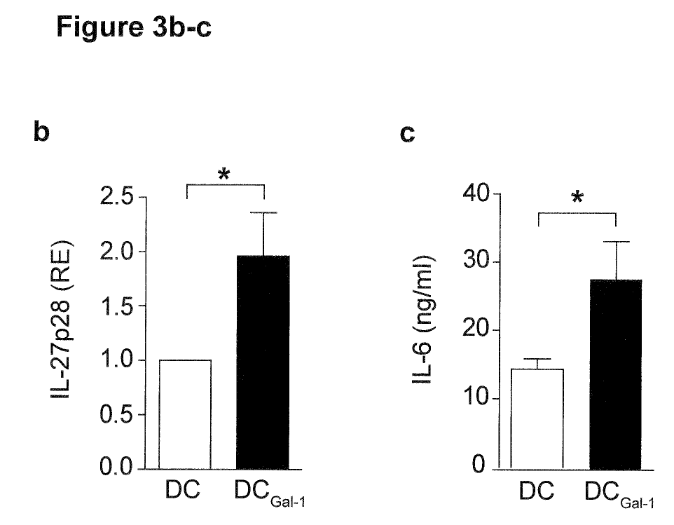

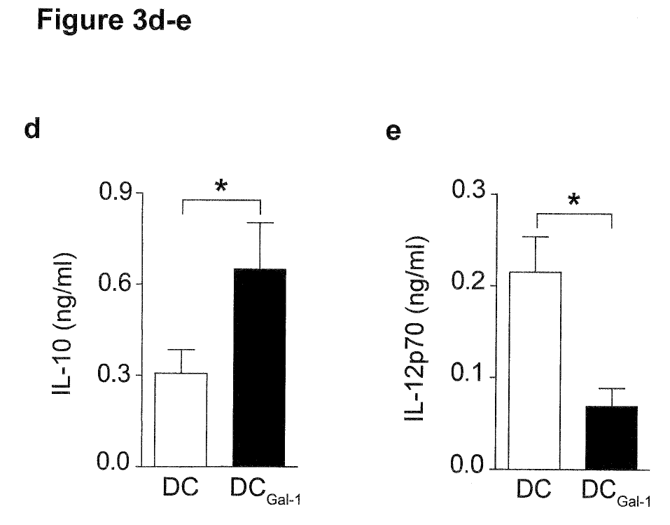

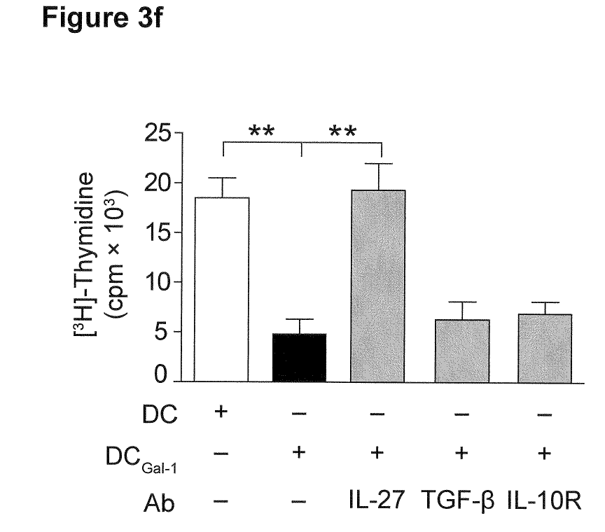

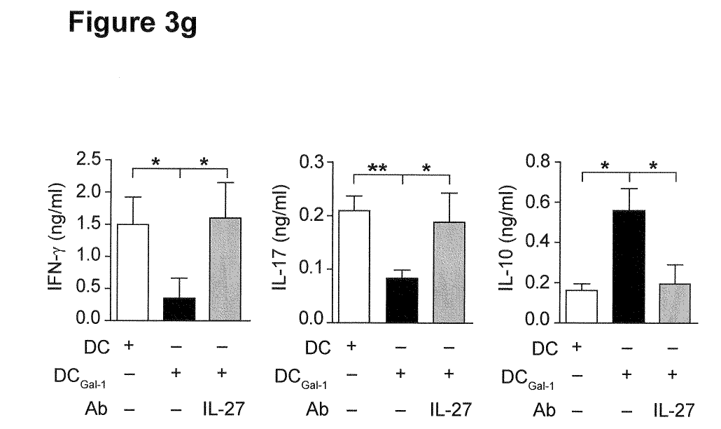

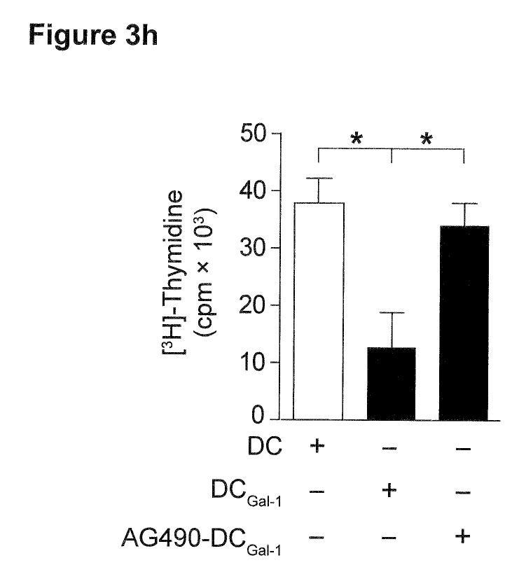

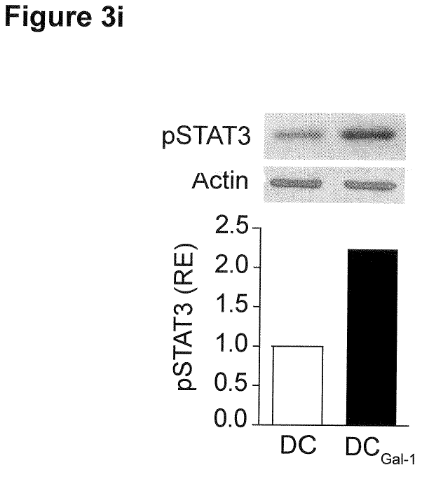

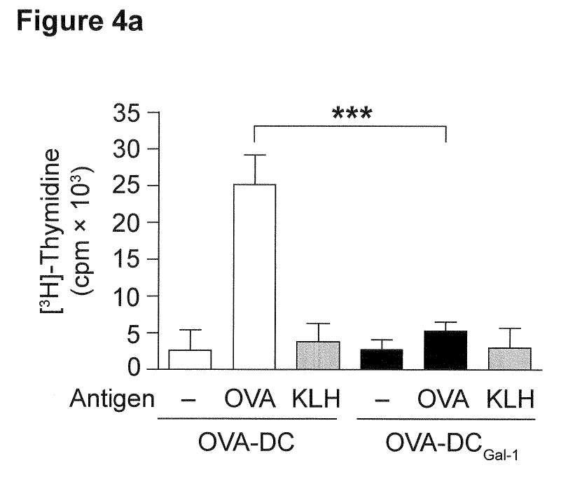

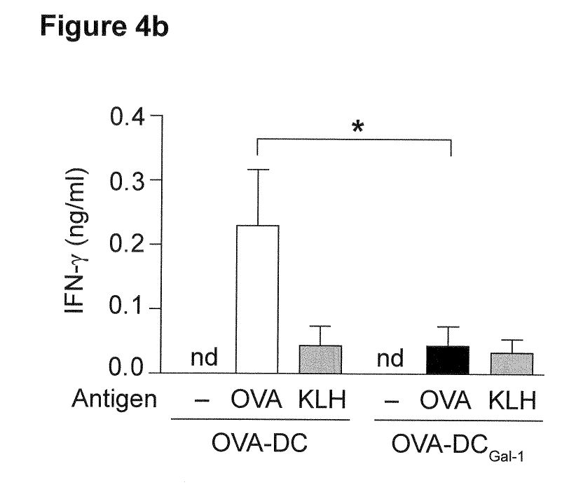

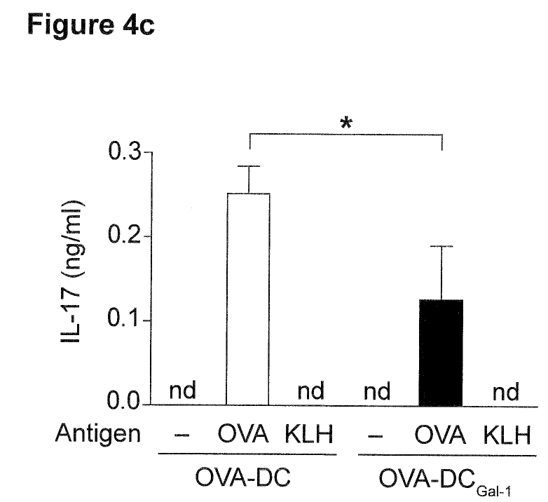

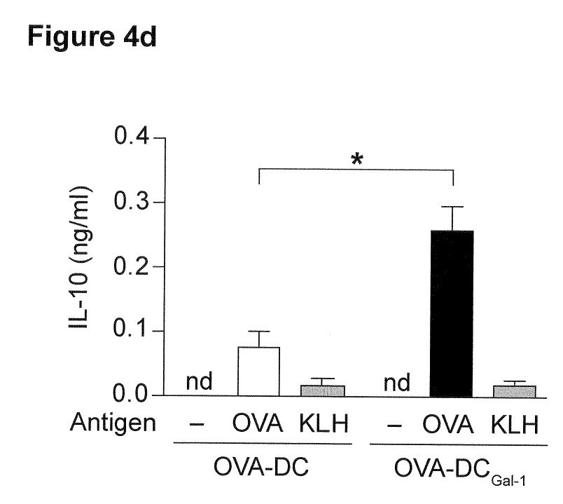

Given the critical regulatory role of the JAK2-STAT3 and NF-KB pathways in APC maturation and function^{3,27,28}, we further explored their contribution to galectin-1 induction of regulatory DCs. Maturation of DCs in the presence of galectin-1 resulted in increased phosphorylation of STAT3 compared to DCs exposed to LPS alone (FIG. 2d). Consistent with these findings, addition of the JAK2-STAT3 inhibitor AG490 during DC maturation reduced the regulatory capacity of DC_{Gal-1} in a dose-dependent manner (FIG. 2e). In contrast, we could find no modulation of the NF-KB pathway, as exposure to galectin-1 during DC maturation did not affect IKBα degradation, nor NF-κB DNA-binding activity, as compared to DCs matured with LPS alone (Supplementary FIGS. 3a,b online). Thus, galectin-1 endows human DCs with regulatory potential through modulation of the JAK2-STAT3 signaling pathway. Notably, we could observe no considerable variations in the frequency of apoptotic or viable cells along the cell culture when human DCs were exposed to galectin-1, either during differentiation or maturation of these cells (Supplementary FIG. 4a-d online).

Collectively, our results indicate that, in the presence of inflammatory stimuli, galectin-1 drives the generation of human DCs with a mature phenotype but greatly enhanced regulatory potential.

### Galectin-1 Programs the Differentiation of IL-27-Producing Mouse Tolerogenic DCs

Because galectin-1 is a candidate for the induction of regulatory DCs with tolerogenic potential in vivo, we analyzed the impact of this glycan-binding protein on the differentiation of mouse DCs from bone marrow cells cultured in the presence of GM-CSF. Addition of galectin-1 at day 0 of the cell culture induced the differentiation of a population of mouse DCs (DC_{Gal-1}) with low expression of CD11c and costimulatory molecules and high expression of CD45RB, a cell surface marker associated with regulatory DCs^{6,11} (FIG. 3a). In contrast, differentiation of bone marrow cells in the presence of GM-CSF alone resulted in the generation of CD11c^{hi}CD45RB⁻ DCs. Differentiation of CD11c^{lo}CD45RB⁺ DCs by galectin-1 relied on protein-glycan interactions, as it was prevented by addition of the specific disaccharide lactose (FIG. 3a, right panels). Moreover, upon further maturation with LPS, both DC populations adopted a similar cell surface phenotype of fully mature DCs (data not shown).

Interleukin-27, a member of the IL-12 family, has recently emerged as a dominant cytokine produced by tolerogenic DCs, which acts in conjunction with IL-6 to induce IL-10-producing T cells¹³⁻¹⁵. Remarkably, we found considerable up-regulation of IL-27 in CD11c^{lo}CD45RB⁺ DCs differentiated in the presence of galectin-1 (FIG. 3b), which was sustained upon DC maturation (data not shown). In addition, we detected higher amounts of IL-6 and IL-10 and lower amounts of IL-12 in DC_{Gal-1} compared to DCs differentiated with GM-CSF alone and further matured with LPS (FIG. 3c-e). To examine the functional properties of galectin-1-conditioned mouse DCs, we co-cultured DC_{Gal-1} or control DCs with alloreactive naïve CD4 T cells. Priming with control DCs resulted in vigorous proliferation and synthesis of large amounts of IFN-Y and IL-17 by alloreactive CD4 T cells, whereas CD11c^{lo}CD45RB⁺ DC_{Gal-1} induced only weak proliferation of alloreactive T cells and negligible synthesis of IFN-γ and IL-17 (FIGS. 3f,g). However, CD4 T cells co-cultured with DC_{Gal-1} secreted substantially higher amounts of IL-10 compared to CD4 T cells primed with control DCs (FIG. 3g). Of note, we found no significant differences in the amounts of TGF-P or IL-4 secreted by allogeneic CD4 T cells (data not shown) or in the frequency of CD4⁺CD25⁺FoxP3⁺ T cells (Supplementary FIG. 2c online) following priming with either DC_{Gal-1} or control DCs.

To further understand the mechanisms involved in the tolerogenic potential of DC_{Gal-1}, we analyzed the contribution of IL-27, IL-10 and TGF-β to the regulatory function of these cells. Remarkably, blockade of IL-27 using an IL-27p28-specific monoclonal antibody completely eliminated the regulatory capacity of DC_{Gal-1} on CD4 T cell proliferation and cytokine (IFN-γ, IL-17 and IL-10) secretion (FIGS. 3f,g). However, neutralization of TGF-β or blockade of IL-10 receptor, using specific monoclonal antibodies, could not reverse the ability of DC_{Gal-1} to suppress allogeneic T cell responses (FIG. 3f). Thus, CD11c^{lo}CD45RB⁺DCs generated in the presence of galectin-1 are endowed with an IL-27-dependent, but IL-10- and TGF-β-independent regulatory function. Of note, incorporation of an isotype control antibody to MLR stimulated with DC_{Gal-1} had no detectable effect (data not shown). Furthermore, incorporation of the JAK2-STAT3 inhibitor AG490 during DC maturation partially abrogated the regulatory function of DC_{Gal-1} (FIG. 3h), consistent with the ability of galectin-1 to trigger STAT3 phosphorylation on mouse DCs (FIG. 3i). Collectively our data underscore a role for galectin-1-saccharide lattices in driving the differentiation of CD11c^{lo}CD45RB⁺ tolerogenic DCs, which favor the induction of IL-10-producing T cells through IL-27- and STAT3-dependent mechanisms.

### Galectin-1-Differentiated DCs Induce Antigen-Specific Tolerance In Vivo

To determine whether DCs differentiated in a galectin-1-enriched microenvironment have enhanced regulatory function in vivo, we pulsed DC_{Gal-1} (CD11c^{lo}CD45RB⁺IL-27^{hi}) or control DCs (CD11c^{hi}CD45RB⁻IL-27^{lo}) overnight with OVA and then transferred these cells into syngeneic naïve mice. Seven days after challenge, we immunized mice with OVA in complete Freund's adjuvant (CFA). We then analyzed antigen-specific proliferation and cytokine production in splenocytes of mice given autologous DC_{Gal-1} or control DCs following ex vivo restimulation with OVA. We found vigorous antigen-specific proliferation and large amounts of IFN-γ and IL-17 production in splenocytes of mice given OVA-pulsed control DCs prior to immunization (FIG. 4a-c). In contrast, adoptive transfer of OVA-pulsed DC_{Gal-1} markedly suppressed antigen-specific proliferation and inhibited IFN-γ and IL-17 secretion by splenocytes of recipient mice (FIG. 4a-c). However, we found greatly enhanced secretion of IL-10 by splenocytes of mice given OVA-pulsed DC_{Gal-1} compared to those given OVA-pulsed control DCs (FIG. 4d). This effect was antigen-specific as it was not detected when we restimulated splenocytes ex vivo with an unrelated antigen such as keyhole limpet hemocyanin (KLH) (FIG. 4a-d). Moreover, we observed no effects when mice received OVA-pulsed DCs and were further challenged with KLH or when mice were given unpulsed DC_{Gal-1} or control DCs prior to immunization (data not shown). Our results indicate that galectin-1 imparts a regulatory program which facilitates the generation of regulatory DCs with antigen-specific tolerogenic function in vivo.

### Galectin-1-Differentiated DCs Favor T Cell Tolerance at Tumor Sites

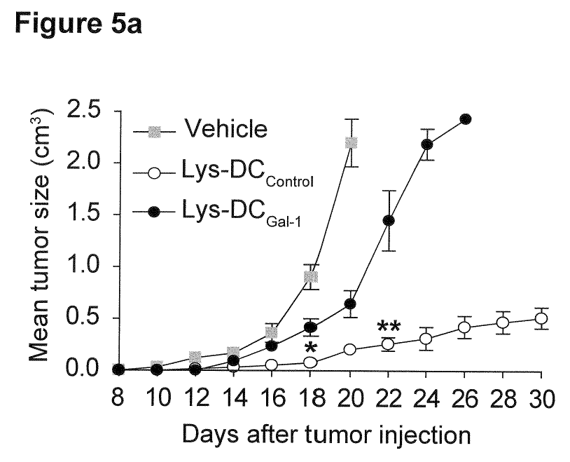

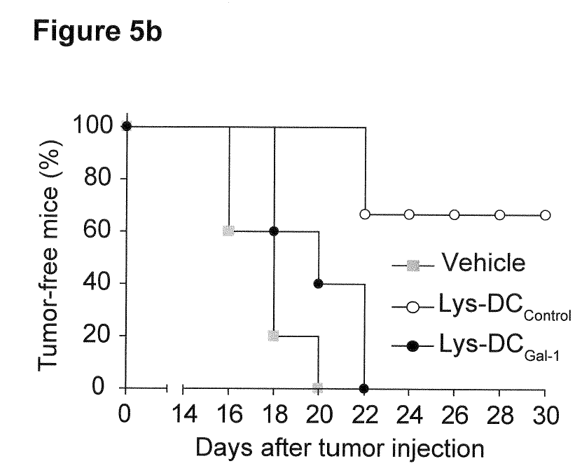

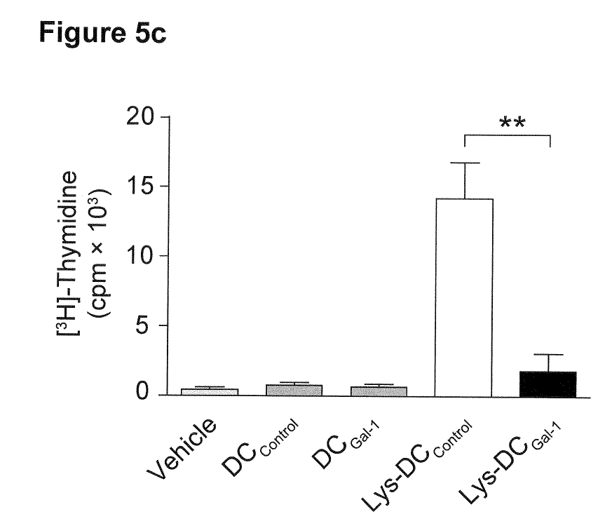

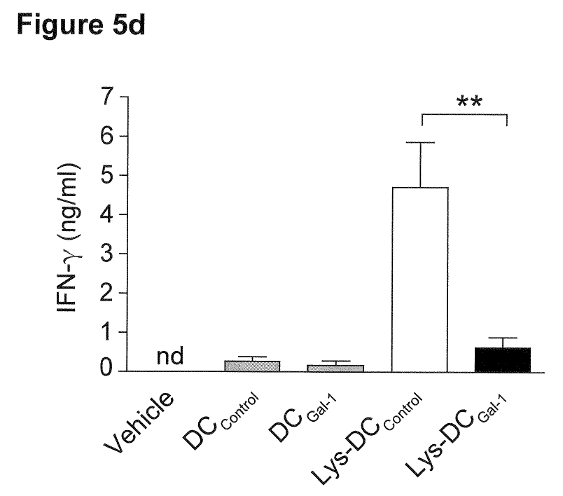

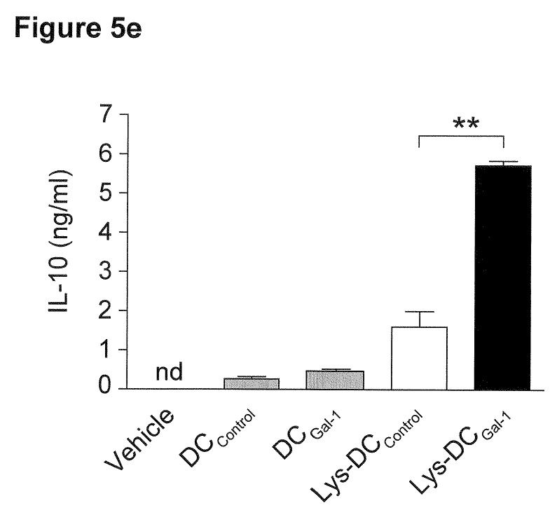

Tumor lysate-pulsed DCs can elicit effective antitumor responses and protect mice against challenge with viable tumor cells²⁹. However, the protective function of DCs could be thwarted by immunosuppressive factors found at tumor sites²⁵. To investigate the regulatory function of DC_{Gal-1} in an in vivo setting of pathophysiological relevance, we pulsed DC_{Gal-1} (CD11c^{lo}CD45RB⁺IL-27^{hi}) and control DCs (CD11c^{hi}CD45RB⁻IL-27^{lo}) with tumor lysates of B16 melanoma cells and performed tumor protection assays. We immunized B6 mice twice at 7 d intervals with tumor-pulsed or unpulsed DC_{Gal-1} or control DCs, challenged mice 14 d later with viable B16 cells and monitored tumor progression as described²². When we immunized mice with fully competent tumor lysate-pulsed DCs, tumor growth was inhibited by ≧80%, compared with tumors of mice receiving vehicle control or unpulsed DCs before tumor challenge (FIGS. 5a and Supplementary FIG. 5a online). However, we found no substantial inhibition of tumor growth when mice were immunized with tumor lysate-pulsed DC_{Gal-1} (FIG. 5a). Remarkably, all mice immunized with DC_{Gal-1} developed progressively enlarging tumors when challenged with viable melanoma cells at a rate similar to that of mice receiving unpulsed DCs, leading to uniform terminal morbidity by about 20-25 d post-challenge (FIGS. 5b and Supplementary FIG. 5b online). In contrast, 60% of mice immunized with fully competent tumor lysate-pulsed control DCs remained tumor free for about 30 d post-inoculation (FIG. 5b). Thus, tumor lysate-pulsed DCs are not fully competent for protecting against challenge with B16 melanoma cells when they are differentiated in a galectin-1-enriched microenvironment.

To examine the mechanisms underlying this lack of protection, we analyzed the proliferative response and cytokine profile in tumor-draining lymph node cells of mice immunized with DC_{Gal-1} or control DCs and further challenged with viable B16 melanoma cells, following ex vivo restimulation with irradiated B16 cells. Two weeks after tumor challenge lymph node cells from mice immunized with fully competent tumor-pulsed DCs showed a robust proliferative response (FIG. 5c) and produced high amounts of IFN-γ and discrete amounts of IL-10 (FIGS. 5d,e). In contrast, lymph node cells from mice immunized with tumor-pulsed DC_{Gal-1} showed poor proliferative response, reduced synthesis of IFN-γ and greatly enhanced production of IL-10 (FIG. 5c-e). Consistent with the lack of effect on tumor growth, we found no differences in the amount of secreted cytokines when mice were immunized with unpulsed DCs (FIG. 5c-e).

Collectively, our results indicate that, regardless of their maturation status, DCs differentiated in a galectin-1-enriched microenvironment cannot elicit an effective antitumor response against tumor challenge, but instead tilt the cytokine balance to foster a tolerant milieu at tumor sites.

### Therapeutic Effect of DC_{Gal-1} in T_{H}1- and T_{H}-17-Mediated Neuroinflammation

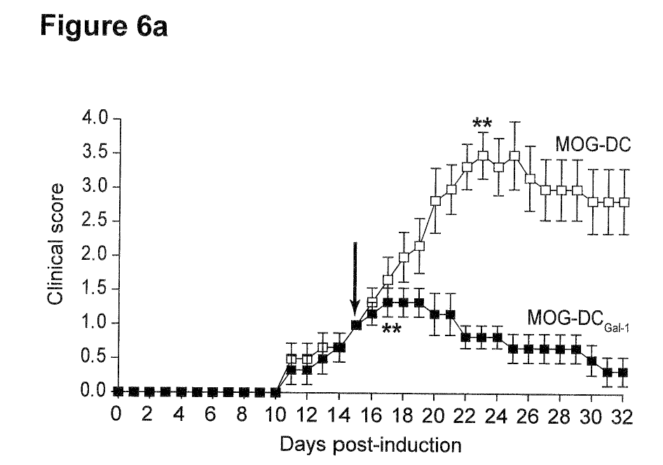

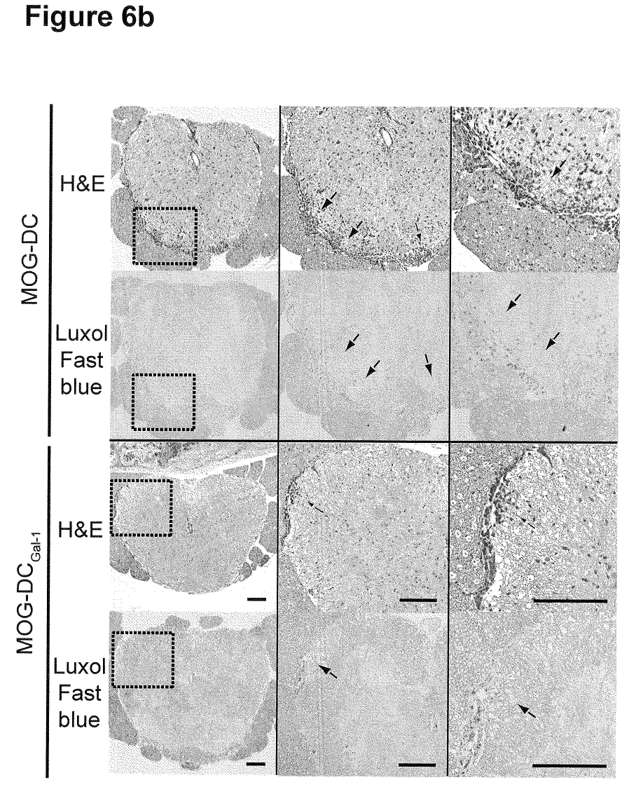

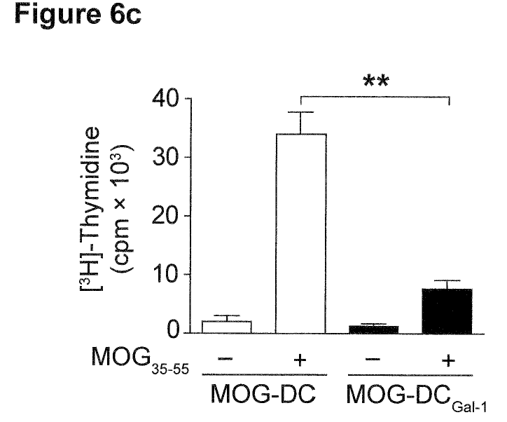

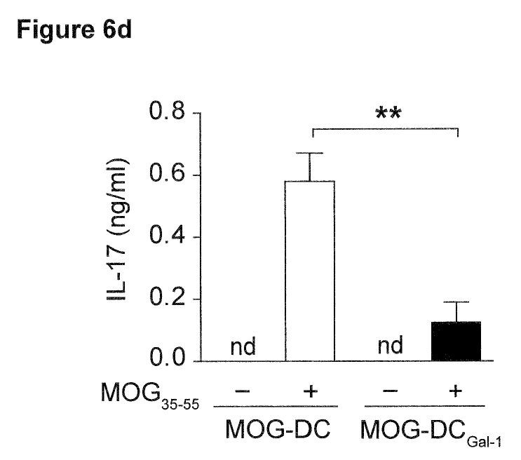

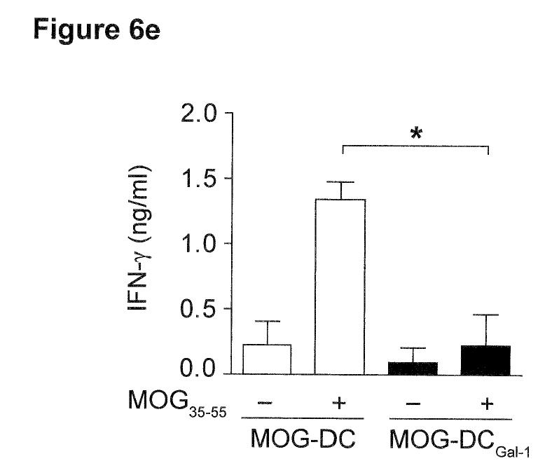

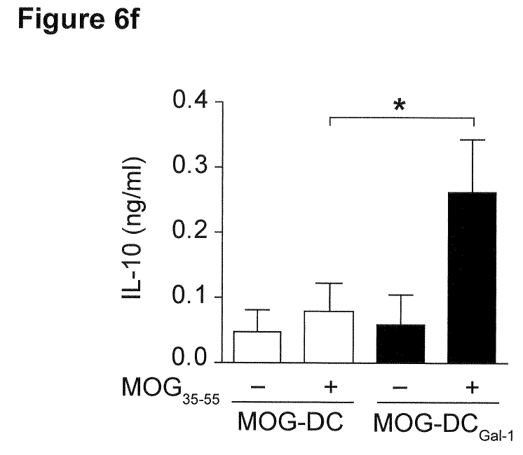

Recent studies proposed the potential use of regulatory DCs as a therapeutic tool to halt organ-specific autoimmune diseases^{4,5,7}. Therefore, we examined the therapeutic effect of IL-27-producing DC_{Gal-1} in experimental autoimmune encephalomyelitis (EAE), a T cell-mediated demyelinating disorder widely used as a model of multiple sclerosis³⁰ We immunized mice with the encephalitogenic peptide (amino acids 35-55) of myelin oligodendrocyte glycoprotein (MOG₃₅₋₅₅) and monitored disease progression. At the day of disease onset (clinical score 1), we injected mice with syngeneic MOG₃₅₋₅₅-pulsed DCs or DC_{Gal-1}. Treatment with MOG₃₅₋₅₅-pulsed DC_{Gal-1} resulted in greatly reduced clinical severity compared to mice treated with MOG₃₅₋₅₅-pulsed control DCs (FIG. 6a and Supplementary Table 1 online). In addition; areas of inflammation and demyelination were much less pronounced in spinal cord sections from mice treated with peptide-pulsed DC_{Gal-1}, compared to those injected with peptide-pulsed control DCs (FIG. 6b). However, we observed no clinical benefits when mice were treated with unpulsed DC_{Gal-1} (Supplementary FIG. 6a online). Importantly, comparable numbers of carboxyfluorescein diacetate succinimidyl ester (CFSE)-labeled control DCs and DC_{Gal-1} reached draining lymph nodes of treated mice, suggesting lack of an effect of galectin-1 on the migratory pattern of DCs in vivo (Supplementary FIG. 6b online).

T_{H}-17 and T_{H}1 effector cells provide distinct but essential contributions to autoimmune neuroinflammation^{30,31}. To understand the mechanistic bases of the anti-inflammatory effect of DC_{Gal-1}, we evaluated MOG₃₅₋₅₅-specific proliferation and cytokine production of lymph node cells from mice treated with peptide-pulsed DC_{Gal-1}. Lymph node cells from mice treated with peptide-pulsed DC_{Gal-1} showed markedly reduced antigen-specific proliferation than did lymph node cells from mice treated with peptide-pulsed control DCs (FIG. 6c). More importantly, therapeutic administration of peptide-pulsed DC_{Gal-1} resulted in greatly diminished MOG₃₅₋₅₅-specific production of IL-17 and IFN-γ and substantially higher amounts of IL-10, compared with mice treated with peptide-pulsed control DCs (FIG. 6d-f). The tolerogenic effect of DC_{Gal-1} was peptide-specific since unpulsed or OVA-pulsed DC_{Gal-1} showed only a weak effect in EAE progression (Supplementary FIG. 6a and data not shown). These results indicate that IL-27-producing DC_{Gal-1} can limit the severity of organ-specific autoimmune inflammation when transferred during established EAE by dampening antigen-specific T_{H}1 and T_{H}17 responses and up-regulating IL-10-producing T cells.

### Endogenous Galectin-1 Fine-Tunes the Tolerogenic Function of DCs

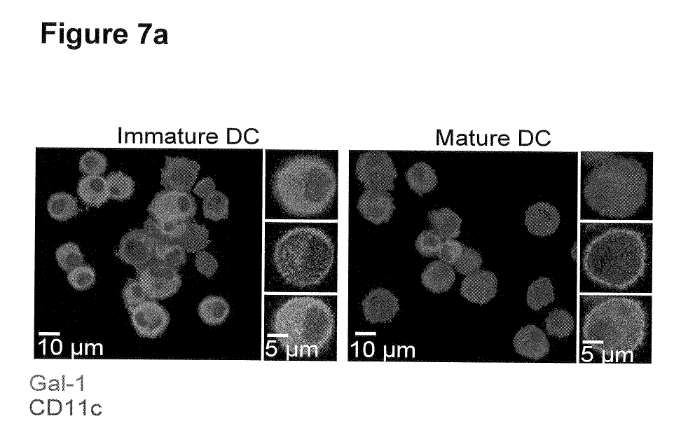

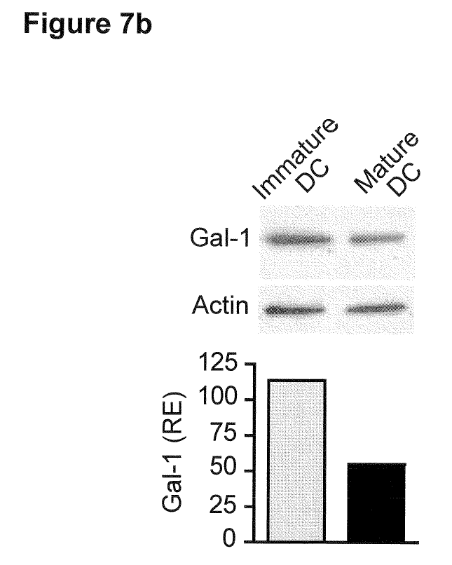

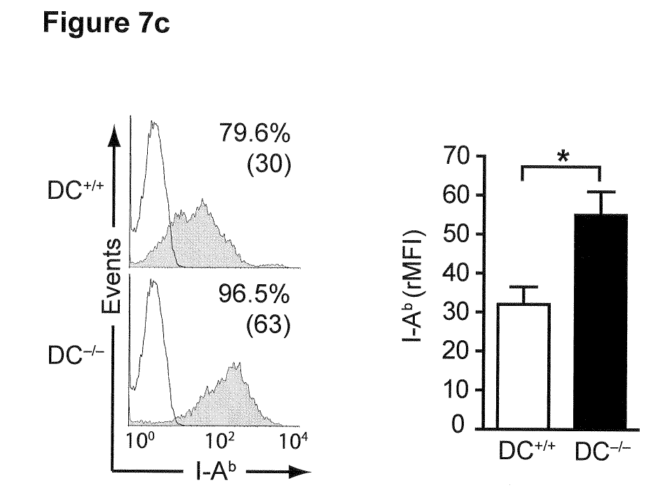

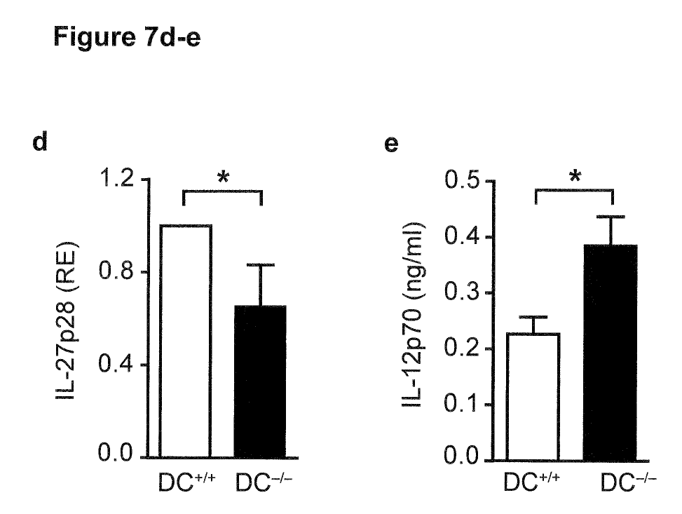

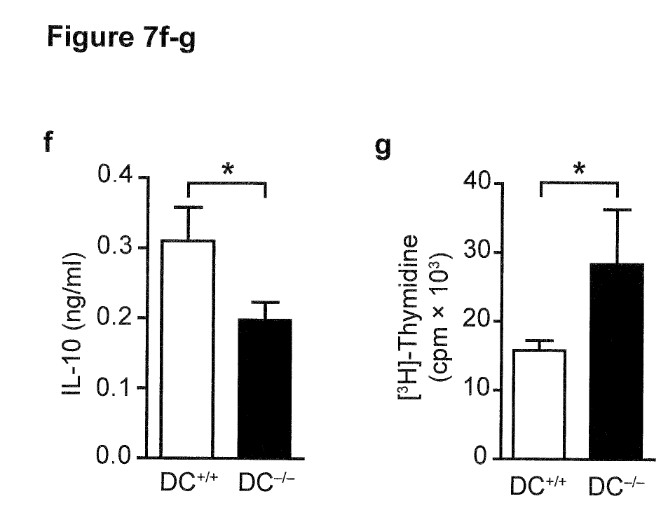

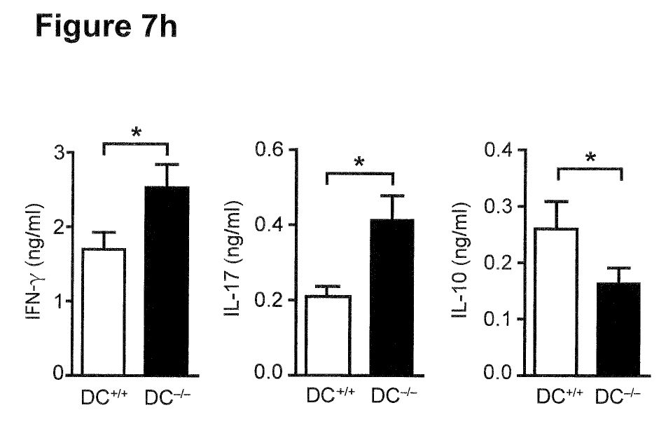

Given the enhanced susceptibility of Lgals-1^{-/-} mice to autoimmune inflammation²¹, we next wished to examine the role of endogenous galectin-1 in the tolerogenic function of DCs. For this, we first analyzed the expression and subcellular compartmentalization of galectin-1 during DC maturation. Bone marrow-derived immature DCs synthesized large amounts of galectin-1, which substantially declined upon maturation with LPS (FIGS. 7a,b). While immature DCs showed an intense staining mainly localized at the cytosolic and perinuclear compartments, mature DCs showed less prominent and more diffuse labeling (FIG. 7a).

To investigate the effect of Lgals1 gene deletion on DC functionality, we differentiated bone marrow cells from Lgals1^{-/-} mice and wild-type littermates in the presence of GM-CSF. Although Lgals1^{-/-} and wild-type DCs displayed a comparable phenotype in terms of most cell surface markers including CD11c and costimulatory molecules (data not shown), Lgals1^{-/-} DCs showed considerably higher MHC II (I-A^{b}) expression compared to wild-type DCs (FIG. 7c). Moreover, Lgals1^{-/-} DCs showed reduced expression of IL-27 (FIG. 7d), and upon maturation synthesized more IL-12 and less IL-10 than their wild-type counterparts (FIGS. 7e,f). Given this skewed cytokine profile, we then examined the allostimulatory capacity and immunogenicity of Lgals1^{-/-} DCs. Allogeneic CD4 T cells stimulated with Lgals1^{-/-} DCs showed more robust proliferation (FIG. 7g) and secreted larger amounts of IFN-γ and IL-17 than allogeneic CD4 T cells primed with wild-type DCs (FIG. 7h). In addition, allogeneic CD4 T cells primed with Lgals1^{-/-} DCs synthesized less IL-10 than those primed with wild-type DCs (FIG. 7h). Of note, we found no differences in the viability of Lgals1^{-/-} and wild-type DCs when differentiated with GM-CSF or matured in the presence of LPS (data not shown).

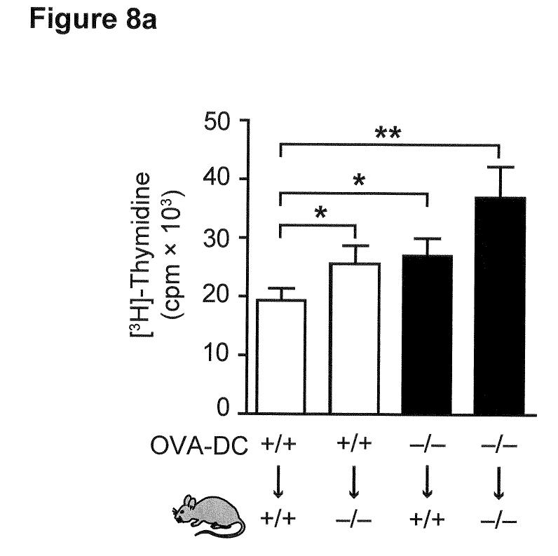

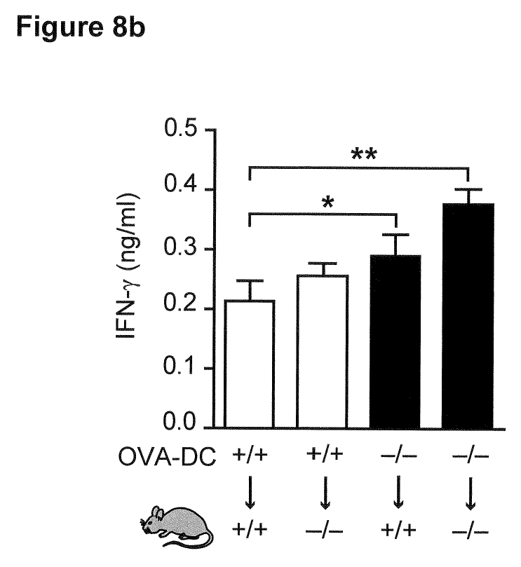

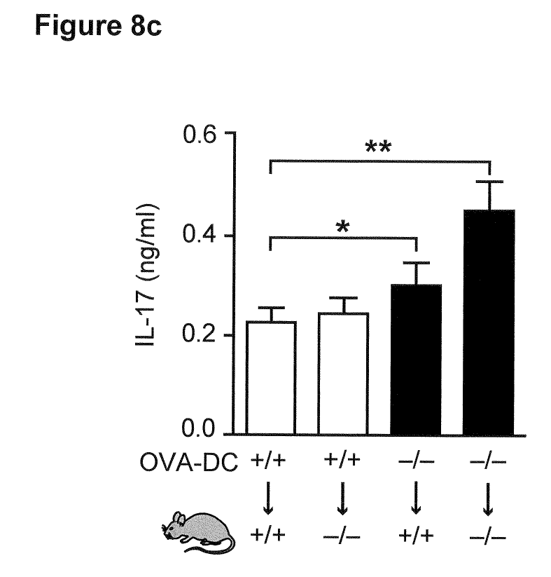

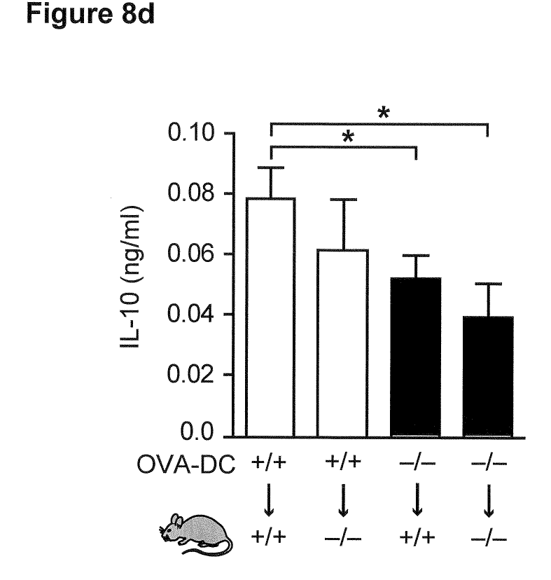

To analyze the immunogenicity of Lgals1^{-/-} DCs in vivo, we pulsed Lgals1^{-/-} or wild-type DCs with OVA and adoptively transferred these cells into Lgals1^{-/-} or wild-type recipient mice. Seven days after challenge, we immunized mice with OVA in CFA. We then analyzed antigen-specific proliferation and cytokine production in splenocytes of mice given Lgals1^{-/-} or wild-type DCs following ex vivo restimulation with OVA, Remarkably, transfer of antigen-pulsed Lgals1^{-/-} DCs resulted in enhanced T cell proliferation (FIG. 8a), increased IFN-γ and IL-17 production (FIGS. 8b,c) and reduced synthesis of IL-10 (FIG. 8d), when adoptively transferred into either Lgals1^{-/-} or wild-type recipients, as compared to OVA-pulsed wild-type DCs transferred into wild-type or Lgals1^{-/-} mice. Collectively, these data hint to a regulatory role of endogenous galectin-1 in fine-tuning the immunogenic or tolerogenic function of DCs.

### Discussion

Recent efforts toward decoding the glycosylation signature of immune cell processes have revealed dramatic changes in N- and O-glycan structures during T-cell activation, differentiation and homeostasis^{21,32-35}. These pronounced alterations have also been detected during the course of DC differentiation and maturation²⁶, suggesting that protein-glycan interactions may have a decisive role in the control of immune cell responsiveness and tolerance¹⁶. Here we have identified an essential role for galectin-1-glycan interactions in the generation of human and mouse regulatory DCs. Galectin-1-differentiated DCs dampened T_{H}-17 and T_{H}1 responses through IL-27-dependent and STAT3-mediated mechanisms, induced antigen-specific tolerance in vivo in inflammatory and neoplastic settings and terminated T_{H}-17- and T_{H}1-mediated neuroinflammation. In addition, we uncovered a novel role of endogenous galectin-1 as a fine-tuner of the tolerogenic function of DCs.

Emerging evidence indicates that endogenous glycan-binding proteins, particularly C-type lectin receptors (CLRs), may serve as signaling molecules which relay pathogen, tumor or self-antigen information into distinct DC differentiation programs³⁶⁻⁴¹. Activation of the CLR DC-SIGN by human immunodeficiency virus-1 induces an immature DC phenotype characterized by increased Rho-GTPase activity³⁷. Furthermore, dectin-1, a CLR that recognizes β-glucan structures on yeasts, signals DCs through the kinase Syk and the adaptor CARD9 to enhance the secretion of IL-23 and drive the differentiation of T_{H}-17 cells³⁸. However, dectin-1 can also mediate the effects of zymosan on the induction of IL-10-producing regulatory DCs³⁹. Similarly, interaction of P-selectin with P-selectin glycoprotein ligand-1 induces the generation of indoleamine 2,3-dioxygenase-producing tolerogenic DCs⁴⁰ and engagement of the CLR Dcir triggers an inhibitory signal to limit DC expansion and functionality⁴¹. Thus, distinct protein-glycan systems may have evolved as ‘on-and-off’ switch programs that control the induction of tolerogenic or immunogenic DCs with critical implications in immune system homeostasis. Here we found that galectin-1-saccharide lattices can signal DCs to produce TL-27 and IL-6 and promote tolerance in vivo through induction of IL-10-producing Tr1 cells. Of interest, van Vliet et al reported that tolerogenic DCs selectively express the CLR MGL to suppress T cell activation³³; hence galectin-1 might also exploit this pathway by inducing MGL-producing tolerogenic DCs, suggesting a link between galectins and C-type lectins in the induction of immune tolerance.

The mechanisms underlying the anti-inflammatory activity of galectin-1¹⁷⁻²¹ remain poorly understood. Although this protein appears to modulate the survival of activated or terminally-differentiated T cells^{21,42} and skew the T_{H}1/T_{H}2 cytokine balance^{19,43}, these mechanisms do not broadly support the immunosuppressive effects observed at early or late phases of the inflammatory response^{17-19,21} In search for alternative mechanisms, we found that galectin-1 triggers a cascade of immunosuppressive events leading to the differentiation of IL-27-producing regulatory DCs which promote antigen-specific T cell tolerance. Although recent work suggested that very high concentrations of galectin-1 (20 μM) can induce a maturation phenotype in vitro when added alone to mouse immature DCs⁴⁴, we demonstrate here that galectin-1, at much lower concentrations (0.3-3 μM), can license human and mouse DCs with tolerogenic potential when added during the differentiation or maturation process along with cytokines or LPS, thus resembling the physiological conditions of DC priming in vivo. One possible explanation for these apparent discrepancies could be a bifunctional role of galectin-1 acting as a tolerogenic signal at physiologically plausible concentrations, but triggering DC maturation when released at high concentrations from the cytosolic compartment of damaged cells. Nevertheless, the bimodal paradigm of fully mature DCs eliciting adaptive immunity as opposed to immature DCs acting as promoters of T-cell tolerance has recently been challenged, indicating that DC maturation per se is neither a distinguishing feature of immunogenic as opposed to tolerogenic DCs nor a control point for initiating immunity^{2,4}. In this regard, we found that exposure to galectin-1 during the maturation process drove the generation of DCs with a mature or semi-mature phenotype, but heightened regulatory potential in vivo. Furthermore, Lgas11^{-/-} DCs had greatly enhanced immunogenic capacity, providing an unequivocal evidence of the tolerogenic, but not immunogenic function of galectin-1 within the DC compartment. Accordingly, we recently found that progesterone-regulated galectin-1 can restore tolerance in failing pregnancies and this effect correlates with a T_{H}2-skewed cytokine profile, expansion of regulatory T cells and the appearance of mucosal uterine cells with a DC regulatory phenotype²⁴.

Several different mechanisms have been proposed to explain the ability of DCs to drive T_{H}1, T_{H}2 and T_{H}-17 differentiation programs². However, the nature of specialized DCs dedicated to selectively halt T_{H}1, T_{H}2 or T_{H}-17 effector immunity is uncertain. In this regard, recent studies underscored a dominant function for IL-27-producing CD11c^{lo}CD45RB⁺ DCs in the generation of IL-10⁺FoxP3⁻ anti-inflammatory T cells^{13-15,45}. Here we showed that galectin-1 imparts a regulatory program on DCs which recapitulates this tolerogenic phenotype leading to the induction of IL-10-producing T cells through IL-27-dependent mechanisms. These cells suppressed antigen-specific T_{H}-17 and T_{H}1 responses and limited the severity of autoimmune neuroinflammation. Given that IL-27-producing DCs can be Generated by close contact with inducible FoxP3+Tregs¹³ which are a major source of galectin-1⁴⁶, we postulate that this glycan-binding protein might represent an elusive immunosuppressive signal which links Treg-induced immunosuppression, IL-27-producing tolerogenic DCs and IL-10-producing anti-inflammatory Tr1 cells. Interestingly, IL-27Rα (WSX-1)-deficient mice develop exacerbated EAE owing to hyper-T_{H}-17 responses⁴⁵ and Lgals1^{-/-} mice completely recapitulate this phenotype²¹.

Galectin-1 recognizes multiple galactose-β1-4-N-acetylglucosamine (LacNAc) units, which may be presented on the branches of N- or O-linked glycans¹⁶. Therefore, the regulated expression of glycosyltransferases during DC differentiation, maturation and function, creating poly-LacNAc ligands²⁶, may determine susceptibility to galectin-1. Consistent with a regulatory function of galectin-glycan lattices on APC physiology, interruption of β1,6 branching on N-glycans by targeted deletion of N-acetylglucosaminyltransferase 5 or blockade of polylactosamine synthesis by disruption of β1,3-N-acetylglucosaminyltransferase 2, results in altered sensitivity to cytokine signaling and reduced threshold for APC activation^{47,48}. In addition, recent studies demonstrated that ligation of Tim-3, a specific receptor for galectin-9, induces distinct signaling events on DCs and T cells leading to initiation or termination of T_{H}1 immunity^{49,50}. Thus, galectin-glycan lattices may have evolved to regulate APC homeostasis and control their activation, differentiation and signaling.

DC-based vaccination represents a promising approach to harness the specificity and potency of the immune system to combat cancer²⁵. However, this immunotherapeutic strategy may be thwarted by immunosuppressive factors elaborated by tumor cells which might convert otherwise immunogenic into tolerogenic DCs capable of impairing antitumor immunity²⁵. We found that, regardless of their maturation status, DCs differentiated in a galectin-1-enriched microenvironment, are not fully competent for eliciting an effective antitumor response, and instead tilt the cytokine balance to foster a tolerant milieu at sites of tumor growth. Notably, gene and protein expression profiles have recurrently led to the identification of galectin-1 as a major regulatory protein secreted by tumor and stromal cells^{22,23,25}, which have been shown to play a dominant role in directing the differentiation of CD11c^{lo}CD45RB⁺ regulatory DCs^{11,12,25}. Hence, tumor and stromal tissue may drive the differentiation of tolerogenic DCs through secretion of galectin-1, thus providing an alternative explanatory mechanism for the role of galectin-1 in tumor-immune escape^{22,23}.

In addition to the regulatory function of galectin-1-glycoprotein lattices in the control of DC physiology, our studies demonstrate that the stimulatory capacity of DCs and the magnitude of adaptive immunity are critically regulated by ‘endogenous’ galectin-1, as Lgals1^{-/-} DCs had enhanced immunogenic capacity compared with wild-type DCs. These results indicate that endogenous galectin-1 imposes a critical brake that may halt the intrinsic immunogenicity of DCs, suggesting that DCs devoid of galectin-1 might have therapeutic advantages as adjuvants for cancer therapy or infectious processes similar to DCs lacking negative regulatory signals such as SOCS1⁵¹, Dcir and STAT3^{27,28}.

In conclusion, our findings identified a link between galectin-1 signaling, differentiation of CD45RB⁺IL-27⁺ regulatory DCs and termination of T_{H}-17 and T_{H}1-mediated inflammation. Strategies to manipulate galectin-1 expression or signaling in either direction (blockade or stimulation) may be able, therefore, to influence immune tolerance versus activation, a critical decision with profound implications in autoimmunity, transplantation and cancer immunotherapy.

This invention encompasses therapeutic formulations, comprising Galectin-induced tolerogenic DCs and a therapeutical acceptable carrier. Certain therapeutic formulations are single unit dosage forms suitable for parenteral (e.g., subcutaneous, intravenous, bolus injection, intraarterial or intramuscular), mucosal (e.g., sublingual, nasal, vaginal, or rectal) or transdermal administration to a patient. Examples of dosage forms include, but are not limited to: dispersions, suppositories, ointments, powders, patches, aerosols (e.g., nasal sprays or inhalers), gels, liquid dosage forms suitable for mucosal administration to a patient, including suspensions (e.g., aqueous or non-aqueous liquid suspensions, oil-in-water emulsions, or a water-in-oil liquid emulsions) and solutions, liquid dosage forms suitable for parenteral administration to a patient, and sterile solids (e.g., crystalline or amorphous solids) that can be reconstituted to provide liquid dosage forms suitable for parenteral administration to a patient.

The formulation should suit the mode of administration. For example, a formulation may contain ingredients that facilitate delivery of the active ingredient(s) to the site of action.

The composition, shape, and type of a dosage form will vary depending on its use. For example, a dosage form used in the acute treatment of a disease may contain larger amounts of the Galectin-induced tolerogenic DCs than a dosage form used in the chronic treatment of the same disease. Similarly, a parenteral dosage form may contain smaller amounts of one or more of the Galectin-induced tolerogenic DCs it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

In a particular embodiment of the invention the therapeutic formulation is suitable for the parenteral administration to a patient. Preferably, the therapeutic formulation is suitable for subcutaneous, intravenous (including bolus injection), bolus injection, intramuscular, or intraarterial administration to a patient.

Because their administration typically bypasses patients' natural defenses against contaminants, parenteral dosage forms are specifically sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection and water-miscible vehicles such as, but not limited to, ethyl alcohol, polyethylene glycol, and polypropylene glycol.

Another object of the invention is a method of preparing a therapeutic formulation which comprises: incubating dendritic cells (DCs) or dendritic cells progenitors (DCPs) in an incubation medium containing Galectin, wherein said Galectin is in a sufficient amount for obtaining Galectin-induced tolerogenic DCs; and suspending said Galectin-induced tolerogenic DCs in a therapeutical acceptable carrier. Said carrier can be selected from a pharmaceutical acceptable excipient, vehicle and/or diluents. According to the invention, in said incubation medium containing Galectin, said Galectin can be encapsulated in liposomes, nanospheres or cyclodextrins. In another embodiment, Galectin can be free.

Preferably, in the method of preparing the therapeutic formulation of the invention, the incubation medium containing Galectin, contains at least one Galectin selected from Galectin-1 and Galectin-2, and more preferably contains Galectin-1. In one embodiment, the incubation medium contains from about 0.1 to about 10 μM of Galectin-1. In another, the incubation medium contains from 0.3 to 3 μM of Galectin-1.

Following the method of the invention, a Galectin-1-induced tolerogenic DCs thus obtained acquired a regulatory phenotype characterized by IL-27-dependent, STAT3-mediated and CD45RB⁺ IL-10⁺ signatures.

It is another object of the invention a method of treating, managing or preventing a chronic inflammatory disease or disorder, which comprises administering to a patient in need thereof a therapeutically or prophylactically effective amount of the therapeutic formulation comprising Galectin-induced tolerogenic DCs and a therapeutical acceptable carrier. Preferably, the method comprises the administration of said therapeutic formulation along with a specific autoantigen responsible of triggering said disease or disorder.

It is another object of the invention a method of treating, managing or preventing an autoimmune disease or disorder, which comprises administering to a patient in need thereof a therapeutically or prophylactically effective amount of the therapeutic formulation comprising Galectin-induced tolerogenic DCs and a therapeutical acceptable carrier. Preferably, the method comprises the administration of said therapeutic formulation along with a specific antigen responsible of triggering said disease or disorder. Particularly, the autoimmune or inflammatory disease or disorder to be treated can be selected from rheumatoid arthritis, multiple sclerosis, graft-vs-host disease, type I diabetes, psoriasis, autoimmune anemias, Crohn disease, celiac disease, Addison disease and uveitis.

It is another object of the invention a method to suppress T cell responses of a patient in need thereof which comprises administering to said patient and effective amount of the therapeutic formulation comprising Galectin-induced tolerogenic DCs and a therapeutical acceptable carrier.

It is another object of the invention a method to suppress IFN-γ-producing T helper-1 cells and IL-17-producing T helper-17 pathogenic responses of a patient in need thereof which comprises administering to a said patient and effective amount of the therapeutic formulation comprising Galectin-induced tolerogenic DCs and a therapeutical acceptable carrier.

It is another object of the invention a method of suppressing transplant rejection induced by T cells in a patient in need thereof which comprises administering to a said patient and effective amount of the therapeutic formulation comprising Galectin-induced tolerogenic DCs and a therapeutical acceptable carrier. Particularly, the organ to be transplanted can be selected from kidney, liver, heart, pancreas, lung, bone marrow and cornea.

### 6. EXAMPLES

Aspects of this invention can be understood from the following examples, which do not limit its scope.

### Methods

Mice. Galectin-1-deficient (Lgals1^{-/-}) mice (B6) were generated as described⁵². Wild-type B6 and BALB/c mice were obtained from the Faculty of Veterinary Sciences (University of La Plata, Argentina). Mice (6-8-week old) were bred at the animal facility of the Faculty of Exact and Natural Sciences (University of Buenos Aires) according to institutional guidelines. Protocols were approved by the Institutional Review Board of the Institute of Biology and Experimental Medicine (Buenos Aires, Argentina).

Preparation of recombinant galectin-1. The expression and purification of recombinant galectin-1 were accomplished as outlined previously^{17,21}. Potential LPS contamination was carefully removed by Detoxi-Gel™ endotoxin removing gel (Pierce) and tested using with a Gel Clot Limulus Test (<0.5 IU/mg; Cape Code).

Generation of human and mouse DCs. Human DCs were generated from leukopheresis products of healthy blood donors as described⁸. In brief, peripheral blood mononuclear cells (PBMCs) were isolated by density gradient centrifugation on Ficoll-Hypaque™ Plus (GE Healthcare) and monocytes were separated by centrifugation on a discontinuous Percoll gradient (GE Healthcare). The monocyte-enriched population was further purified by positive selection (Monocyte Isolation Kit; Miltenyi Biotec). The purity of CD14⁺ monocytes was checked by flow cytometry (>90w). To obtain immature DCs, monocytes were cultured at 1−1.5×10⁶ cells/ml in complete medium [RPMI 1640 supplemented with 10% heat-inactivated fetal calf serum (FCS), 40 μg/ml gentamicin, 50 μM 2-mercaptoethanol and 2 mM L-glutamine (all from Gibco)] containing 5 ng/ml IL-4 (Sigma) and 35 ng/ml recombinant human GM-CSF (Sigma) in the absence or presence of galectin-1 (iDC_{Gal-1}; 0.3-3 μM). In another set of experiments, cells were differentiated in the absence of galectin-1, but exposed to 1 μg/ml LPS (0111:B4 strain; Sigma) and galectin-1 at day 7 for 24 h.

Bone marrow-derived mouse DCs were generated as described⁷. Briefly, bone marrow cells (1−1.5×10⁶/ml) obtained from Lgals-1^{-/-} or wild-type (B6) mice were incubated in DMEM complete medium supplemented with 20 ng/ml recombinant mouse GM-CSF (BD Biosciences) or 15% conditioned medium from the GM-CSF-producing J588L cells in the absence or presence of galectin-1 (DC_{Gal-1}; 0.3-3 μM). At day 9, more than 90% of the harvested cells expressed CD11c, MHC II and CD86 but not Gr-1. In another set of experiments, DC_{Gal-1} or control DCs were exposed to LPS for further maturation for 48 h. The resulting immature or mature DCs were analyzed for cell surface phenotype, cytokine production and functionality. In selected experiments, the JAK2-STAT3 inhibitor AG490 (Calbiochem) was added to cell cultures. In other experiments, NF-κB p50/p65 DNA-binding activity was determined using the EZ-Transcription Factor Assay (Millipore). Cell death was checked at different time points by staining with fluorescein isothyocyanate (FICT)-conjugated annexin V (BD Biosciences) and cell viability was determined by Trypan blue dye exclusion as described²¹.

Galectin-1 binding and segregation assays. Cells (5×10⁵) were incubated for 1 h at 4° C. with biotinylated galectin-1 in the presence or absence of increasing concentrations of lactose or sucrose as described²¹. Cells were then incubated for 45 min at 4° C. with FITC-conjugated streptavidin (Pierce), washed and analyzed in a FACSAria™. (BD Biosciences). Nonspecific binding was determined using FITC-conjugated streptavidin alone. For analysis of CD43 and CD45 segregation, DCs (2×10⁶) were treated with galectin-1 or buffer control for 1 h, were fixed for 30 min at 4° C. with 2% (wt/vol) paraformaldehyde and were incubated for 1 h with mouse monoclonal antibody to human CD43 (8.4 μg/ml; DF-T1; Dako) or to human CD45 (14.5 μg/ml; 2B11; Dako) followed by FITC-conjugated anti-mouse immunoglobulin G (F0479; Dako) and counterstaining with propidium iodide (10 μg/ml; Sigma). Cells showing receptor segregation were analyzed on a Nikon laser confocal microscope (Eclipse E800).

Flow cytometry. Cells were incubated for 30 min at 4° C. with various FITC- and phycoerythrin (PE)-labeled monoclonal antibodies (all from BD Biosciences). Human cells were stained with FITC-anti-CD1a (HI149), PE-anti-CD14 (M5E2), FITC-anti-CD86 (2331-FUN-1), FITC-anti-HLA-DR (G46-6), PE-anti-CD83 (HB15e) monoclonal antibodies. Mouse cells were stained with PE-anti-CD11c (HL3), FITC-anti-CD40 (HM40-3), PE-anti-I-A^{b} (AF6-120.1), PE-H-2K^{b} (AF6-88.5), FITC-anti-CD80 (16-10A1), FITC-anti-CD86 (GL1), FITC-anti-Gr1 (RB6-8C5) and FITC-anti-CD45RB (16A) monoclonal antibodies. Nonspecific binding was determined using appropriate fluorochrome-conjugated, isotype-matched irrelevant antibodies. For intracellular staining, cells were processed as described², made permeable with Fix & Perm reagents (Caltag) and stained with an anti-FoxP3 antibody (FJK-16s; eBioscience). Data were acquired on a FACSAria™ (BD Biosciences).

Endocytosis assay. Differentiated cells (1×10⁶) were suspended in culture medium in the presence of 300 μg/ml FITC-OVA (Sigma) for 30 min at 37° C. Control DCs were pulsed with FITC-OVA at 4° C. After extensive washing, cells were analyzed on a FACSAria™ (BD Biosciences).

Cytokine assays. Cytokine contents in the supernatants of DCs, allogeneic MLR cultures and lymph node cells were analyzed by enzyme-linked immunosorbent assays (ELISAs) using capture/biotinylated detection antibodies. The human and mouse IL-12p70, IL-10, IFN-γ, IL-6, and TGF-β₁ ELISA sets were from BD Biosciences and the mouse IL-17 ELISA kit was from R&D.

Real-time quantitative RT-PCR. Total RNA from DCs (5×10⁶) was prepared using Trizol (Invitrogen) as described⁶. The real-time quantitative PCR was performed with the SYBR Green PCR Master Mix (Applied Biosystem) in an ABI PRISM 7500 Sequence Detection Software (Applied Biosystem) according to the manufacturer's instructions. PCR (1 cycle: 95° C. 10 min, 40 cycles: 95° C. 15 sec, 60° C. 1 min) was used to quantify mRNA. Primers used were: IL-27p28: forward 5′-ATCTCGATTGCCAGGAGTGA, reverse: 5—GTGGTAGCGAGGAAGCAGAGT. GAPDH expression was used as internal control.

Allogeneic MLR. Human CD4 T cells were purified from PBMCs of healthy donors by negative selection using the CD4 T Cell Isolation kit (RosetteSep™; StemCell Technol). Human control DCs or DC_{Gal-1} were washed with complete medium, irradiated (3,000 rad) and co-cultured with allogeneic CD4 T cells (1×10⁵) at various DC:T ratios for 5 d. To determine whether DC_{Gal-1}, had regulatory potential, human allogeneic CD4 T cells (2×10⁵) were co-cultured for 5 d with LPS-matured fully-competent DCs (1×10⁴) in the absence or presence of variable numbers of DC_{Gal-1}. MLR cultures were then analyzed for proliferation and cytokine production.

Mouse naïve CD4 T cells (CD62L+CD44^{lo}) were isolated from spleens of BALB/c mice with the MagCellect Isolation kit (R&D). DCs differentiated in the absence or presence of galectin-1 or generated from Lgals1^{-/-} mice were washed, irradiated (3,000 rad) and co-cultured for 5-6 d with naïve CD4 splenocytes (2×10⁵) from BALB/c mice at various T:DCs ratios. Proliferation was assessed by [³H]-thymidine incorporation (1 μCi/well; specific activity 5 Ci/mM; DuPont) for the final 18 h of culture. In selected experiments the anti-IL-27p28 (AF1834; R&D), anti-TGF-β (1D11; R&D), or anti-IL-10 receptor (CD210; 1B1.3a; BD Biosciences) neutralizing antibodies were added at the beginning of MLR.

Adoptive transfer experiments. Wild-type DCs, DC_{Gal-1} or Lgals1^{-/-}DCs were pulsed with OVA (200 μg/ml; Sigma) for 24 h and adoptively transferred (3×10⁵/mouse) into syngeneic wild-type or Lgals1^{-/-} recipient mice by intraperitoneal injection. At day 7 after transfer, these mice were immunized subcutaneously with OVA in CFA. To examine the immunogenic effect of transferred DCs, 7 d later splenocytes were obtained and analyzed for antigen-specific T cell proliferation and cytokine production following ex vivo culture for 72 h in the absence or presence of OVA (75 μg/ml).

Immunoblot and immunofluorescence analyses. Immunoblot analysis was done as described 22. Equal amounts of protein were resolved by SDS-PAGE, blotted onto nitrocellulose membranes (GE Healthcare) and probed with anti-STAT3 (H-190), anti-pSTAT3 (B-7), anti-IκBα (C-21), anti-actin (I-19) antibodies (all from Santa Cruz) or with a rabbit anti-galectin-1 immunoglobulin G (1.5 μg/ml) generated and used as described^{22,24}. Bound antibodies were detected with peroxidase-labeled anti-rabbit immunoglobulin G (170-6515; BioRad), followed by development with enhanced chemoluminescence (GE Healthcare). Films were analyzed with Scion Image software.

For immunofluorescence labeling, cells (2×10⁶) were fixed in 1t (wt/vol) paraformaldehyde for 15 min, blocked with 10% (vol/vol) goat serum, 1% (wt/vol) BSA, permeabilized with Perm-2 solution (BD Biosciences) and stained with a rabbit anti-galectin-1 immunoglobulin G (32 μg/ml) followed by a FITC-labeled anti rabbit immunoglobulin G (sc-2012; Santa Cruz) and a PE-labeled CD11c-specific antibody (20 μg/ml; HL-3; BD Biosciences). Nonspecific binding was determined using a rabbit preimmune immunoglobulin G or isotype control. Cells were analyzed on a Nikon laser confocal microscope (Eclipse E800).

Tumor protection assays. B16 melanoma cells were suspended at 1×10⁶ cells/ml and subjected to four cycles of rapid freeze/thaw exposures for preparation of lysates as described²⁹. B6 mice were immunized twice subcutaneously at 7-d intervals with tumor-lysate-pulsed or unpulsed DCs or DC_{Gal-1} (1×10⁶). After 14 d of the last immunization, mice were challenged subcutaneously with 2×10⁵ viable B16 melanoma cells. Tumor development was monitored every second day by measuring tumor perpendicular diameters as described²². Mice with tumor volume less than 0.5 cm³ were considered as tumor free for the Kaplan-Meier analysis. For ethical reasons, animals were sacrificed when tumors reached a volume greater than 2.5 cm³. Tumor-draining lymph nodes were isolated from each group of mice two weeks after tumor challenge. Lymph node cells (5×10⁵/well) were restimulated ex vivo with 1×10⁴ irradiated (4000 rads) B16 cells for 72 h and analyzed for proliferation and cytokine production.

Induction and assessment of EAE. Therapeutic protocol. EAE was induced in 6- to 8-week old female mice (B6) by subcutaneous immunization with 300 μg MOG₃₅₋₅₅ (MEVGWYRSPFSRVVHLYRNGK; Sigma) in CFA supplemented with 4 mg/ml of M. tuberculosis (H37Ra; Difco). Mice received 200 ng pertussis toxin (List Biological Labs) on days 0 and 2. Mice were examined daily for signs of EAE and were assigned scores as follows: 1, limp tail; 2, hindlimb weakness; 3, hindlimb paralysis; 4, hindlimb and forelimb paralysis; and 5, moribund. Mice with established EAE (clinical score 1) were injected intraperitoneally with 2×10⁵ syngeneic MOG₃₅₋₅₅-pulsed or unpulsed DCs or DC_{Gal-1}. On days 25-30, spinal cords were fixed in 10′ (vol/vol) formalin and paraffin-embedded sections 6 μm in thickness were stained with hematoxylin and eosin and with Luxol Fast blue. Migration of pulsed or unpulsed DC_{Gal-1} or DC_{control} was checked by labeling the cells with CFSE (5 μM; Molecular Probes) before transfer. Proliferation of antigen-specific splenocytes was assessed by incorporation of [³H]-thymidine after ex vivo restimulation with MOG₃₅₋₅₅. Cytokine production was analyzed by ELISA in splenocytes after 72 h of antigen restimulation as described²¹.

### Statistical Analysis.

Comparison of two groups was made using the Student's t test for unpaired data when appropriate using Prism software (GraphPad). Kaplan-Meier analysis was used to establish statistical significance for tumor protection assays. The Mann-Whitney U test was used for clinical scores. P values of 0.05 or less were considered significant.

### REFERENCES

1. Guermonprez, P., Valladeau, J., Zitvogel, L., Thery, C. & Amigorena, S. Antigen presentation and T cell stimulation by dendritic cells. Annu. Rev. Immunol. 20, 621-667 (2002).
2. Reis e Sousa, C. Dendritic cells in a mature age. Nat. Rev. Immunol. 6, 476-483 (2006).
3. Steinman, R. M., Hawiger, D. & Nussenzweig, M. C. Tolerogenic dendritic cells. Annu. Rev. Immunol. 21, 685-711 (2003).
4. Rutella, S., Danese, S. & Leone, G. Tolerogenic dendritic cells: cytokine modulation comes of age. Blood 108, 1435-1440 (2006).
5. Morelli, A. E. & Thomson, A. W. Tolerogenic dendritic cells and the quest for transplant tolerance. Nat. Rev. Immunol. 7, 610-621 (2007).
6. Wakkach, A. et al. Characterization of dendritic cells that induce tolerance and T regulatory 1 cell differentiation in vivo. Immunity 18, 605-617 (2003).
7. Chorny, A. et al. Vasoactive intestinal peptide induces regulatory dendritic cells with therapeutic effects on autoimmune disorders. Proc. Natl. Acad. Sci. USA 102, 13562-13567 (2005).
8. Rutella, S. et al. Hepatocyte growth factor favors monocyte differentiation into regulatory interleukin (IL)-10+IL-12 low/neg accessory cells with dendritic-cell features. Blood 108, 218-227 (2006).
9. Penna, G. et al. 1,25-Dihydroxyvitamin D3 selectively modulates tolerogenic properties in myeloid but not plasmacytoid dendritic cells. J. Immunol. 178, 145-153 (2007).
10. Skoberne, M. et al. The apoptotic-cell receptor CR3, but not αvβ5, is a regulator of human dendritic-cell immunostimulatory function. Blood 108, 947-955 (2006).
11. Svensson, M., Maroof, A., Ato, M. & Kaye, P. M. Stromal cells direct local differentiation of regulatory dendritic cells. Immunity 21, 805-816 (2004).
12. Zhang, M. et al. Splenic stroma drives mature dendritic cells to differentiate into regulatory dendritic cells. Nat. Immunol. 5, 1124-1133 (2004).
13. Awasthi, A. et al. A dominant function for interleukin 27 in generating Interleukin 10-producing anti-inflammatory T cells. Nat. Immunol. 8, 1380-1389 (2007).
14. Stumhofer, J. S. et al. Interleukins 27 and 6 induce STAT3-mediated T cell production of interleukin 10. Nat. Immunol. 8, 1363-1371 (2007).
15. Fitzgerald, D. C. et al. Suppression of autoimmune inflammation of the central nervous system by interleukin 10 secreted by interleukin 27-stimulated T cells. Nat Immunol. 8, 1372-1379 (2007).
16. van Kooyk, Y. & Rabinovich, G. A. Protein-glycan interactions in the control of innate and adaptive immune responses. Nat. Immunol. Submitted (2008).
17. Rabinovich, G. A. et al. Recombinant galectin-1 and its genetic delivery suppress collagen-induced arthritis via T cell apoptosis. J. Exp. Med. 190, 385-398 (1999).
18. Santucci, L. et al. Galectin-1 suppresses experimental colitis in mice. Gastroenterology 124, 1381-1394 (2003).
19. Toscano, M. A. et al. Galectin-1 suppresses autoimmune retinal disease by promoting concomitant Th2- and T regulatory-mediated anti-inflammatory responses. J. Immunol. 176, 6323-6332 (2006).
20. Perone, M. J. et al. Dendritic cells expressing transgenic galectin-1 delay onset of autoimmune diabetes in mice. J. Immunol. 177, 5278-5289 (2006).
21. Toscano, M. A. et al. Differential glycosylation of T_{H}1, T_{H}2 and T_{H}-17 effector cells selectively regulates susceptibility to cell death. Nat. Immunol. 8, 825-834 (2007).
22. Rubinstein, N. et al. Targeted inhibition of galectin-1 gene expression in tumor cells results in heightened T cell-mediated rejection; A potential mechanism of tumor-immune privilege. Cancer Cell 5, 241-251 (2004).
23. Juszczynski, P. et al. The AP1-dependent secretion of galectin-1 by Reed Sternberg cells fosters immune privilege in classical Hodgkin lymphoma. Proc. Natl. Acad. Sci. USA 104, 13134-13139 (2007).
24. Blois, S. M. et al. A pivotal role for galectin-1 in fetomaternal tolerance. Nat. Med. 13, 1450-1457 (2007).
25. Rabinovich, G. A., Gabrilovich, D. & Sotomayor, E. M. Immunosuppressive strategies that are mediated by tumor cells. Annu. Rev. Immunol. 25, 267-296 (2007).
26. Bax, M. et al. Dendritic cell maturation results in pronounced changes in glycan expression affecting recognition by siglecs and galectins. J. Immunol. 179, 8216-8224 (2007).
27. Cheng, F. et al. A critical role for Stat3 signaling in immune tolerance. Immunity 19, 425-436 (2003).
28. Nefedova, Y. et al. Regulation of dendritic cell differentiation and antitumor immune response in cancer by pharmacologic-selective inhibition of the janus-activated kinase 2/signal transducers and activators of transcription 3 pathway. Cancer Res. 65, 9525-9535 (2005).
29. Kotera, Y., Shimizu, K. & Mule, J. J. Comparative analysis of necrotic and apoptotic tumor cells as a source of antigen(s) in dendritic cell-based immunization. Cancer Res. 61, 8105-8109 (2001).
30. Steinman, L. A brief history of T_{H}-17, the first major revision in the T_{H}1/T_{H}2 hypothesis of T cell-mediated tissue damage. Nat. Med. 13, 139-145 (2007).
31. Stromnes, I. M., Cerretti, L. M., Liggitt, D., Harris, R. A. & Goverman, J. M. Differential regulation of central nervous system autoimmunity by T_{H}1 and T_{H}-17 cells. Nat. Med. 14, 337-42 (2008).
32. Comelli, E. M. et al. Activation of murine CD4⁺ and CD8⁺ T lymphocytes leads to dramatic remodeling of N-linked glycans. J. Immunol. 177, 2431-2440 (2006).
33. van Vliet, S. J., Gringhuis, S. I., Geijtenbeek, T. B. & van Kooyk, Y. Regulation of effector T cells by antigen-presenting cells via interaction of the C-type lectin MGL with CD45. Nat. Immunol. 7, 1200-1208 (2006).
34. Morgan, R. et al. N-acetylglucosaminyltransferase V (Mgat5)-mediated N-glycosylation negatively regulates Th1 cytokine production by T cells. J. Immunol. 173, 7200-7208 (2004).
35. Van Dyken, S. J., Green, R. S. & Marth, J. D. Structural and mechanistic features of protein O-glycosylation linked to CD8⁺ T-cell apoptosis. Mol. Cell. Biol. 27, 1096-1111 (2007).
36. Robinson, M. J., Sancho, D., Slack, E. C., LeibundGut-Landmann, S. & Reis e Sousa, C. Myeloid C-type lectins in innate immunity. Nat. Immunol. 7, 1258-1265 (2006).
37. Hodges, A. et al. Activation of the lectin DC-SIGN induces an immature dendritic cell phenotype triggering Rho-GTPase activity required for HIV-1 replication. Nat. Immunol. 8, 569-577 (2007).
38. LeibundGut-Landmann, S. et al. Syk- and CARD9-dependent coupling of innate immunity to the induction of T helper cells that produce interleukin 17. Nat. Immunol. 8, 630-638 (2007).
39. Dillon, S. et al. Yeast zymosan, a stimulus for TLR2 and dectin-1, induces regulatory antigen-presenting cells and immunological tolerance. J. Clin. Invest. 116, 916-928 (2006).
40. Urzainqui, A. et al. Functional role of P-selectin glycoprotein ligand 1/P-selectin interaction in the generation of tolerogenic dendritic cells. J. Immunol. 179, 7457-7465 (2007).
41. Fujikado, N. et al. Dcir deficiency causes development of autoimmune diseases in mice due to excess expansion of dendritic cells. Nat. Med. 14, 176-180 (2008).
42. Stillman, B. N. et al. Galectin-3 and galectin-1 bind distinct cell surface glycoprotein receptors to induce T cell death. J. Immunol. 176, 778-789 (2006).
43. Stowell, S. R. et al. Differential roles of galectin-1 and galectin-3 in regulating leukocyte viability and cytokine secretion. J. Immunol. 180, 3091-3102 (2008).
44. Fulcher, J. A. et al. Galectin-1-matured human monocyte-derived dendritic cells have enhanced migration through extracellular matrix. J. Immunol. 177, 216-226 (2006).
45. Batten, M. et al. Interleukin 27 limits autoimmune encephalomyelitis by suppressing the development of interleukin 17-producing T cells. Nat. Immunol. 7, 929-936 (2006).
46. Garin, M. I. et al. Galectin-1: a key effector of regulation mediated by CD4⁺CD25⁺ T cells. Blood 109, 2058-2065 (2007).
47. Partridge, E. A. et al. Regulation of cytokine receptors by Golgi N-glycan processing and endocytosis. Science 306, 120-124 (2004).
48. Togayachi, A. et al. Polylactosamine on glycoproteins influences basal levels of lymphocyte and macrophage activation. Proc. Natl. Acad. Sci. USA 104, 15829-15834 (2007).
49. Anderson, A. C. et al. Promotion of tissue inflammation by the immune receptor Tim-3 expressed on innate immune cells. Science 318, 1141-1143 (2007).
50. Zhu, C. et al. The Tim-3 ligand galectin-9 negatively regulates T helper type 1 immunity. Nat. Immunol. 6, 1245-1252 (2005).
51. Shen, L., Evel-Kabler, K., Strube, R. & Chen, S. Y. Silencing of SOCS1 enhances antigen presentation by dendritic cells and antigen-specific anti-tumor immunity. Nat. Biotechnol. 22, 1546-1553 (2004).
52. Poirier, F. & Robertson, E. J. Normal development of mice carrying a null mutation in the gene encoding the L14 S-type lectin. Development 119, 1229-1236 (1993).

1. A method of preparing a therapeutic formulation which comprises: incubating dendritic cells (DCs) or dendritic cells progenitors (DCPs) in an incubation medium containing Galectin, wherein said Galectin is in a sufficient amount for obtaining Galectin-induced tolerogenic DCs; and suspending said Galectin-induced tolerogenic DCs in a therapeutical acceptable carrier.

2. The method of preparing a therapeutic formulation of claim 1, wherein said therapeutical acceptable carrier is a pharmaceutical acceptable excipient, vehicle and/or diluents.

3. The method of preparing a therapeutic formulation of claim 1, wherein in said incubation medium containing Galectin, said Galectin is encapsulated in liposomes, nanospheres or cyclodextrins.

4. The method of preparing a therapeutic formulation of claim 1, wherein said incubation medium containing Galectin, contains at least one Galectin selected from Galectin-1 and Galectin-2.

5. The method of preparing a therapeutic formulation of claim 4, wherein said incubation medium containing Galectin, contains Galectin-1.

6. The method of preparing a therapeutic formulation of claim 5, wherein said incubation medium containing Galectin, contains from about 0.1 to about 10 μM of Galectin-1.

7. The method of preparing a therapeutic formulation of claim 5, wherein said incubation medium containing Galectin, contains from about 0.3 to 3 μM of Galectin-1.

8. The method of preparing a therapeutic formulation of claim 5, wherein said Galectin-1-induced tolerogenic DCs acquired a regulatory phenotype characterized by IL-27-dependent, STAT3-mediated and CD45RB⁺IL-10⁺ signatures.

9. A therapeutic formulation, comprising Galectin-induced tolerogenic DCs and a therapeutic acceptable carrier.

10. The therapeutic formulation of claim 9 comprising Galectin-1-induced tolerogenic DCs and a pharmaceutical acceptable carrier.

11. The therapeutic formulation of claim 9 further comprising pharmaceutical acceptable excipients, vehicles and/or diluents.

12. The therapeutic formulation of claim 9, suitable for mucosal, parenteral or transdermal administration to a patient.

13. The therapeutic formulation of claim 12, suitable for subcutaneous, intravenous, bolus injection, intramuscular or intraarterial administration to a patient.

14. The therapeutic formulation of claim 13, wherein said carrier or said vehicle is an aqueous vehicle or water for injection.

15. The therapeutic formulation of claim 13, wherein said carrier or said vehicle is a water-miscible vehicle.

16. A method of treating, managing or preventing a chronic inflammatory disease or disorder, which comprises administering to a patient in need thereof a therapeutically or prophylactically effective amount of the therapeutic formulation of claim 9.

17. A method of treating, managing or preventing a chronic inflammatory disease or disorder, which comprises administering to a patient in need thereof a therapeutically or prophylactically effective amount of the therapeutic formulation of claim 9 and a specific autoantigen responsible of triggering said disease or disorder.

18. A method of treating, managing or preventing an autoimmune disease or disorder, which comprises administering to a patient in need thereof a therapeutically or prophylactically effective amount of the therapeutic formulation of claim 9.

19. A method of treating, managing or preventing an autoimmune disease or disorder, which comprises administering to a patient in need thereof a therapeutically or prophylactically effective amount of the therapeutic formulation of claim 9 and a specific antigen of said disease or disorder.

20. The method of claim 16, wherein the autoimmune or inflammatory disease or disorder is rheumatoid arthritis, multiple sclerosis, graft-vs-host disease, type I diabetes, psoriasis, autoimmune anemias, Crohn disease, celiac disease, Addison disease or uveitis.

21. A method to suppress T cell responses of a patient in need thereof which comprises administering to a said patient and effective amount of the therapeutic formulation of claim 9.

22. A method to suppress IFN-γ-producing T helper-1 cells and IL-17-producing T helper-17 pathogenic responses of a patient in need thereof which comprises administering to a said patient and effective amount of the therapeutic formulation of claim 9.

23. A method of suppressing transplant rejection induced by T cells in a patient in need thereof which comprises administering to a said patient and effective amount of the therapeutic formulation of claim 9.

24. The method of suppressing transplant rejection of claim 23 wherein the organ to be transplanted is selected from kidney, liver, heart, pancreas, lung, bone marrow and cornea.
